(19) United States
(12) Patent Application Publication
(10) Pub. No.: US 2022/0105362 A1
(43) Pub. Date: Apr. 7, 2022

(54) RE-ACTIVATABLE RADIATION SOURCE FOR BRACHYTHERAPY

(71) Applicant: UNIVERSITY OF IOWA RESEARCH FOUNDATION, IOWA CITY, IA (US)

(72) Inventors: Ryan T. Flynn, Iowa City, IA (US); QUENTIN E. ADAMS, Iowa City, IA (US); KAROLYN M. HOPFENSPERGER, lowa City, IA (US)

(21) Appl. No.: 17/428,035

(22) PCT Filed: Feb. 11, 2020
(86) PCT No.: PCT/US2020/017655

§ 371 Date: Aug. 3, 2021

Related U.S. Application Data
(60) Provisional application No. 62/803,986, filed on Feb. 11, 2019.

(51) Int. Cl. A61N 5/10

(52) U.S. Cl. CPC A61N 5/1007; A61N 2005/1008; A61N 2005/1022

(57) ABSTRACT

A method can comprise irradiating an active element having an initial total activity between zero and thirty curies of ytterbium-169 and a volume between about two cubic millimeters and about four cubic millimeters. Irradiation can be ceased before the active element surpasses a total activity of thirty curies and an activity concentration of ten curies per cubic millimeter.

### CROSS-REFERENCE TO RELATED APPLICATION

This application claims priority to and the benefit of U.S. Provisional Patent Application No. 62/803,986, filed Feb. 11, 2019, the entirety of which is hereby incorporated by reference herein.

### GOVERNMENT SUPPORT CLAUSE

This invention was made with government support under a Phase I STTR grant (R41 CA210737 01) and R01 grant (R01 EB020665), awarded by National Institutes of Health. The government has certain rights in the invention.

### FIELD

This application relates generally to radiation sources and, specifically, to ytterbium-169 radiation sources that are configured for cost minimization.

### BACKGROUND

Ytterbium active sources can be used for treating a tumor or cancerous area. Conventionally, it is taught that minimizing the size of ytterbium active elements is preferred. One reason is that the cost of the precursor to create ytterbium-169 is high. Moreover, the active source is typically physically placed at or near a target via a catheter, and active elements having large diameters cannot fit through off-the-shelf catheters, in particular those placed interstitially. Similarly, when positioned, the off-the-shelf catheters have curved paths, so long active elements cannot fit through the catheters at the curves. Accordingly, the active elements typically have a volume that is less than two cubic millimeters. Moreover, conventional active elements comprising ytterbium-169 have activity concentrations of greater than ten curies per cubic millimeter.

### SUMMARY

Disclosed herein, in one aspect, is a re-activatable radiation source for brachytherapy.

The re-activatable radiation source can be a ytterbium-169 source comprising an active element having a volume between about two cubic millimeters and about four cubic millimeters. The active element can comprise between zero and thirty curies of ytterbium-169 at a start of an activation. At an end of the activation, the active element can have a total activity of less than thirty curies and an activity concentration of less than ten curies per cubic millimeter.

The active element can have a length of between about 7.5 millimeters and about 10.5 millimeters.

The active element can have a length of between about 9 millimeters and about 10.5 millimeters.

The active element can have a length of between about 7.5 millimeters and about 9 millimeters.

The active element can have a diameter between about 0.60 and 0.69 millimeters.

The active element can have a diameter between about 0.60 and 0.65 millimeters.

The active element can have a diameter between about 0.65 and 0.69 millimeters.

The active element can have a diameter that is greater than 0.7 millimeters.

The active element can have a volume between 2.5 and 4 cubic millimeters.

The active element can have a volume between 2.8 and 4 cubic millimeters.

The active element can have a volume between 2.8 and 3.5 cubic millimeters.

The active element can have a volume between 2.5 and 3.5 cubic millimeters.

The active element can have a volume between 2.8 and 3.2 cubic millimeters.

The active element can have a volume between 3 and 4 cubic millimeters.

The active element can have a volume between 3.5 and 4 cubic millimeters.

The ytterbium-169 source can further comprise a radiation source capsule and a radiation source wire. The active element can be disposed within the radiation source capsule. The radiation source wire can be coupled to the radiation source capsule.

The radiation source wire can be configured to be controlled by a remote afterloader.

An applicator can comprise a ytterbium-169 source. The applicator can be a needle.

A method can comprise using the ytterbium-169 source as disclosed herein in a brachytherapy treatment.

In a method for replacing a first active source with a second active source in a source assembly, the source assembly comprising a capsule having a hollow interior and an opening, a first active source disposed therein, and a retainer covering the opening and welded to the capsule, the method can comprise: removing the weld connecting the retainer to the capsule, removing the retainer from the capsule, removing the first active source from the capsule, inserting a second active source in the capsule, replacing the retainer in the capsule, and welding the retainer to the capsule.

The retainer can comprise a plug and a receiver that is configured to couple to the capsule via weldment.

A method can comprise irradiating an active element having an initial total activity between zero and thirty curies of ytterbium-169 and a volume between about two cubic millimeters and about four cubic millimeters and ceasing irradiation before the active element surpasses a total activity of thirty curies and an activity concentration of ten curies per cubic millimeter.

The active element can have a length of between about 7.5 millimeters and about 10.5 millimeters.

The active element can have a length of between about 9 millimeters and about 10.5 millimeters.

The active element can have a length of between about 7.5 millimeters and about 9 millimeters.

The active element can have a diameter between about 0.60 and 0.69 millimeters.

The active element can have a diameter between about 0.60 and 0.65 millimeters.

The active element can have a diameter between about 0.65 and 0.69 millimeters.

The active element can have a diameter that is greater than 0.7 millimeters.

The active element can have a volume between 2.5 and 4 cubic millimeters.

The active element can have a volume between 2.8 and 4 cubic millimeters.

The active element can have a volume between 2.8 and 3.5 cubic millimeters.

The active element can have a volume between 2.5 and 3.5 cubic millimeters.

The active element can have a volume between 2.8 and 3.2 cubic millimeters.

The active element can have a volume between 3 and 4 cubic millimeters.

The active element can have a volume between 3.5 and 4 cubic millimeters.

The method can further comprise positioning the active element within a radiation source capsule and coupling a radiation source wire to the radiation source capsule.

Irradiating the active element can comprise irradiating the active element while the active element is in an inner capsule. Positioning the active element within the radiation source capsule can comprise positioning the inner capsule within the radiation source capsule.

Coupling the radiation source wire to the radiation source capsule can comprise coupling the radiation source wire to the radiation source capsule with a disposable segment that is configured to be cut to decouple the radiation source wire from the radiation source capsule.

The method can further comprise cutting the disposable segment to decouple the radiation source wire from the radiation source capsule.

The method can further comprise establishing communication between the radiation source wire and a remote afterloader.

The method can further comprise reactivating the active element after the step of ceasing irradiation, wherein reactivating the source comprises: irradiating the active source; and ceasing irradiation before the active element surpasses a total activity of thirty curies and an activity concentration of ten curies per cubic millimeter.

A system can comprise an applicator. A catheter can be rotatably disposed within the applicator. A ytterbium-169 source can be disposed within the catheter. The ytterbium-169 source can comprise an active element having a volume between about two cubic millimeters and about four cubic millimeters, wherein the active element has a total activity of less than thirty curies and an activity concentration of less than ten curies per cubic millimeter.

A method can comprise inserting, into an applicator, a ytterbium-169 source, wherein the ytterbium-169 source comprises an active element having a volume between about two cubic millimeters and about four cubic millimeters, wherein the active element has a total activity of less than thirty curies and an activity concentration of less than ten curies per cubic millimeter.

A catheter can be inserted into the applicator prior to inserting the ytterbium-169 source into the applicator.

An afterloader can be used to insert the ytterbium-169 source into the catheter.

The catheter can be rotated with respect to the applicator.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed apparatus, system, and method and together with the description, serve to explain the principles of the disclosed apparatus, system, and method.

FIG. 1 is a ¹⁶⁹Yb source according to embodiments disclosed herein.

FIGS. 2A and 2B are simulated activation and precursor bumup curves for various active source volumes for one year of continuous activation and thirty days of continuous activation, respectively.

FIG. 3A illustrates activity vs. time curves for ¹⁶⁹Yb sources; FIG. 3B illustrates clinical usage years obtained and precursor bumup.

FIG. 4 illustrates resource consumption needed for one year of clinical ¹⁶⁹Yb source generation.

FIGS. 5A-5C illustrate an applicator for cervical cancer.

FIGS. 6A and 6B illustrate radiation dose distributions for the ¹⁶⁹Yb source.

FIG. 7 illustrates dose distributions for various radiation methods.

FIG. 8A illustrates HR-CTV D₉₀-values, including 44 Gy_{EQD2} of external beam radiotherapy dose, for RSBT techniques using either 1 mm³ or 3 mm^{3 169}Yb active sources, for 37 cervical cancer patients. FIG. 8B illustrates delivery times for the delivery techniques of FIG. 8A.

FIGS. 9A-9C illustrate various views of a prostate cancer RSBT needle with ¹⁶⁹Yb sources of two sizes.

FIGS. 10A-10B dose rate distributions for the 3 mm^{3 169}Yb radiation source with an active source length diameter of 0.6 mm and an active source length of 10.5 mm in a prostate cancer RSBT needle.

FIG. 11A shows prostate mean (bars) and standard deviation (whiskers) D₉₀-values for single-shot dose escalation (monotherapy) and urethra D₁₀-values for single-shot urethra-sparing (boost therapy) for 26 patients. FIG. 11B illustrates delivery times for the delivery techniques of FIG. 11A.

FIG. 12A illustrates a cross section of a rotating shield brachytherapy applicator for use with the ¹⁶⁹Yb source of FIG. 1. FIG. 12B illustrates a cross sectional view a portion of the rotating shield brachytherapy applicator of FIG. 12A. FIG. 12C illustrates a side view a catheter of the rotating shield brachytherapy applicator of FIG. 12A. FIG. 12D illustrates a motor drive system for use with the applicator of FIG. 12A. FIG. 12E illustrates another view of a motor drive system for use with the applicator of FIG. 12A. FIG. 12F illustrates yet another view of a motor drive system for use with the applicator of FIG. 12A.

FIG. 13 is a schematic of an exemplary ¹⁶⁹Yb source in accordance with embodiments disclosed herein.

Although some Figures include exemplary dimensions, it should be understood that these dimensions are optional and, therefore, not limiting.

### DETAILED DESCRIPTION

The disclosed system and method may be understood more readily by reference to the following detailed description of particular embodiments and the examples included therein and to the Figures and their previous and following description.

### A. Definitions

It is to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural references unless the context clearly dictates otherwise. Thus, for example, reference to “an active element” includes a plurality of such active elements, and reference to “the active element” is a reference to one or more active elements and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It will be understood that a range from a first value to a second value should include such first and second value and all points therebetween. For example, a range from 0.6 mm to 0.7 mm should include the endpoints 0.6 mm and 0.7 mm. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Introduction

Disclosed herein is a re-activatable radiation source for brachytherapy comprising ytterbium-169, referred to herein as a ¹⁶⁹Yb radiation source. The ¹⁶⁹Yb radiation source for brachytherapy can have an active element volume of 2-4 cubic millimeters and active element activity concentration of 10 curies per cubic millimeter or less. The ¹⁶⁹Yb radiation source can be activated by placing an active source element containing ¹⁶⁸Yb and 0-30 Ci of ¹⁶⁹Yb in a nuclear reactor for a sufficient amount of time to reach an activity that, when delivered to a clinic, is under 30 Ci. The active source can be encapsulated and mounted to a source wire and controlled by a remote afterloader device for use in low-dose-rate brachytherapy (LDR-BT), pulsed-dose-rate brachytherapy (PDR-BT) brachytherapy, medium-dose-rate brachytherapy (MDR-BT), and high-dose-rate brachytherapy (HDR-BT). The source can be re-activated as many times as is practical. Exemplary embodiments of the ¹⁶⁹Yb source can have dimensions such as a diameter between about 0.60 millimeters and about 0.69 millimeters diameter and a length of between about 7.5 millimeters and about 10.5 millimeters, which corresponds to volumes of 2.12-3.96 cubic millimeters.

Brachytherapy can be used to treat various diseases and is particularly effective for treating cervical cancer and prostate cancer. About 13,800 cervical cancer patients and 191,900 prostate cancer patients are expected to be newly diagnosed in 2020 in the U.S. alone.

Rotating shield brachytherapy (RSBT) is a technology with the potential to launch a new era of high-dose-rate brachytherapy (HDR-BT) in which partially-shielded radiation sources, with shields that rotate throughout treatment, enable improved tumor dose conformity, reduced normal tissue doses, or both. The improvement is enabled by the ability to use dose distributions that are deliberately non-symmetric about the brachytherapy applicators for both intracavitary and interstitial brachytherapy. With conventional brachytherapy, especially HDR-BT, dose distributions are radially-symmetric about the applicators and needles, which substantially limit deliverable tumor dose conformity. RSBT technologies have been under development for several years and, through simulation studies, have been shown to have the potential to improve cervical cancer and prostate cancer therapy. Another approach that could improve upon intracavitary HDR-BT is direction-modulated brachytherapy (DMBT), wherein a multi-channel intracavitary applicator with a stationary central shield is used to improve tumor dose conformity and healthy tissue sparing relative to the conventional approach of using a tandem applicator without a central shield.

A major impediment to delivering dose distributions with RSBT and DMBT that are clinically equivalent to or superior to the conventional HDR-BT approach is the lack of a low-cost radiation source with an appropriate size, photon energy spectrum, and dose rate that can replace the conventional ¹⁹²Ir radiation source. For the case of interstitial RSBT, which could benefit prostate cancer patients, ¹⁹²Ir sources emit photons with such high energies that partial shielding within an interstitial needle with a diameter of 2 mm or less provides little to no dosimetric benefit. This is due to the high ¹⁹²Ir photon transmission through a shield of any material, regardless of composition or density. For intracavitary RSBT, which could benefit cervical cancer patients, the thickness of the radiation shield that can fit inside an intracavitary applicator is sufficiently limited that the benefits of the intracavitary RSBT approach may not be sufficient to justify replacement of the conventional, yet more invasive, combined intracavitary/interstitial brachytherapy approach, as is quantitatively demonstrated herein.

The dosimetric benefits of both the intracavitary and interstitial RSBT approaches can be drastically increased if ¹⁶⁹Yb is used as the therapeutic isotope rather than ¹⁹²Ir. U.S. Pat. No. 7,530,941 to John J. Munro, III et al. (hereinafter, “Munro”), which is hereby incorporated by reference for all purposes, discloses a Ytterbium-169 active source. Munro, however, discloses an active source pellet having size of two cubic millimeters or less and a concentration of at least ten curies/cubic millimeter, which can be prohibitively expensive, as will become apparent. As disclosed herein, a ¹⁶⁹Yb source can be geometrically optimized to be cost effective while maintaining the clinically substantial benefit of RSBT for two example treatment sites that constitute a large fraction of those treated with HDR-BT: cervical cancer and prostate cancer. The disclosed ¹⁶⁹Yb source, if not partially-shielded, would provide comparable dose distributions to a conventional ¹⁹²Ir HDR-BT source, thus patients who would not benefit from ¹⁶⁹Yb-based RSBT or DMBT could be treated with applicators that resemble those conventionally used. International Patent Application No. PCT/US2019/052944 to Flynn et al., filed Sep. 25, 2019, entitled “Apparatus and Method for Rotating Shield Brachytherapy,” which is hereby incorporated by reference in its entirety, discloses an exemplary shielded applicator and method of use for the ¹⁶⁹Yb source as disclosed herein. Relevant physical properties for several Ir and Yb isotopes, including half-life and average photon emission energy, are listed in Table 1.

TABLE 1
Isotope properties for Ir and Yb.

| | | | | | Natural | Enriched |
|---|---|---|---|---|---|---|
| Isotope | σ [cm²] | t_{1/2} [d] | λ [s⁻¹] | Ē [keV] | Abundance | Abundance |
| ¹⁹¹Ir | 954 × 10⁻²⁴ | Stable | 0 | N/A | 37.3% | N/A |
| ¹⁹²Ir | 1,420 × 10⁻²⁴ | 73.8 | 1.1 × 10⁻⁷ | 360 | 0% | 0% |
| ¹⁹³Ir | 111 × 10⁻²⁴ | Stable | 0 | N/A | 62.7% | N/A |
| ¹⁶⁸Yb | 2,300 × 10⁻²⁴ | Stable | 0 | N/A | 0.13% | 82% |
| ¹⁶⁹Yb | 3,600 × 10⁻²⁴ | 32 | 2.5 × 10⁻⁷ | 93 | 0% | 0% |
| ¹⁷⁰Yb | 10 × 10⁻²⁴ | Stable | 0 | N/A | 3% | 0% |

Definitions: σ = thermal neutron neutron absorption cross section; t_{1/2} = half-life, λ = ln(2)/t_{1/2} = decay constant, Ē = average emitted photon energy.

### C. Re-Activatable Radiation Source for Brachytherapy

¹⁶⁹Yb can be generated by irradiating a precursor material containing ¹⁶⁸Yb, such as 82% enriched ¹⁶⁸Yb—Yb₂O₃, with thermal neutrons in a nuclear reactor. Throughout the remainder of this disclosure, the referenced precursor material will be 82% enriched ¹⁶⁸Yb—Yb₂O₃, although various other precursor materials can be used, such as metallic Yb, YbF₃, Yb₂(C₂O₄)₃, Yb(NO)₃, Yb₃Si₅, YbCl₃, and Yb₂(SO₄)₃. Naturally-occurring Yb contains only 0.13% ¹⁶⁸Yb, and obtaining a ¹⁶⁹Yb source with a high enough specific activity to enable HDR-BT brachytherapy treatments in a reasonable timeframe of 5-120 minutes can require the activation of enriched precursor material, and ¹⁶⁸Yb enrichment percentages can vary. The cost of 82% enriched ¹⁶⁸Yb—Yb₂O₃ as of Dec. 31, 2018 was $692 per gram according to one supplier, with an example practical density when encapsulated in a brachytherapy source capsule of 8.15 mg/mm³, although other densities are possible. Accordingly, the cost of the precursor material needed to generate a ¹⁶⁹Yb source with a volume of 1-4 mm³ is $5,500-$22,000, and this cost is therefore a major consideration when commercializing the radiation source. The competing conventional isotope, ¹⁹²Ir, can have a negligible precursor cost due to the high abundance of ¹⁹¹Ir (37.5%) in naturally-occurring iridium.

Laser-based enrichment and ceramic production methods can increase the density of the ¹⁶⁸Yb—Yb₂O₃ active source material to greater than 10 g/cm³, increasing the specific activity of the ¹⁶⁹Yb sources by 25%.

According to one aspect, the active source has a volume of 2-4 mm³, larger than previously disclosed ¹⁶⁹Yb radiation sources. The volume of an HDR-BT source is a parameter of high importance since the volume constrains (i) the maximum allowable precursor material in the source, affecting the source activation cost and ability to re-activate the source, (ii) the maximum allowable active source isotope (¹⁶⁹Yb) activity in the source, which directly affects dose rate and therefore treatment times, (iii) the dose distribution about the source, which can degrade treatment plan quality if the source becomes too long, becoming a line source rather than a point source, and (iv) the mechanical ability of the source, which is usually mounted to a wire, to navigate complex curves in the catheters placed inside patients for radiation delivery. These four characteristics must be considered together when defining and optimizing the source volume, which is a non-trivial task. Embodiments disclosed herein are optimized by (i) thoroughly modeling the effects of precursor quantity on the resources required to generate a year's supply of ¹⁶⁹Yb, and (ii) constraining the initial clinical source activity to be less than or equal to 27 Ci, which produces an equivalent dose rate to that of a conventional 10 Ci ¹⁹²Ir source at 1 cm lateral to the source in water. A treatment planning analysis was conducted for 37 cervical cancer and 26 prostate cancer patient datasets to demonstrate a lack of degradation of treatment plan quality at source volumes of 2-4 mm³. Because the dimensions of the disclosed active source are unconventional, the applicators needed for RSBT or DMBT must be designed for receiving such large active sources.

It should be understood from the present disclosure that increasing the source volume from 2 mm³ to 3 mm³ theoretically results in approximately a 26-31% annual cost savings in precursor material and nuclear reactor time, and further increasing the volume from 3 mm³ to 4 mm³ theoretically results in approximately an additional 11-21% cost savings in precursor material and nuclear reactor time. These volume increases come without loss in dosimetric benefit for prostate and cervical cancer RSBT patients.

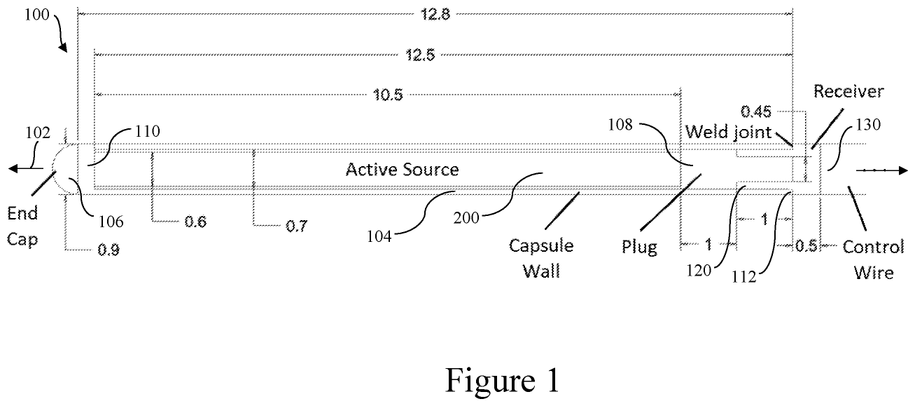

Referring to FIG. 1, an embodiment of a ¹⁶⁹Yb source 100 comprises an active source 200. The source 100 can be elongate about an axis 102. The source 100 can further comprise a capsule 104, hemispherical end cap 106 and a plug 108. The capsule 104 can define a hollow, generally cylindrical body having a closed distal end 110 and an open proximal end 112. The open proximal end 112 can receive the active source 200 therethrough, and the plug 108 can engage a back end of the active source 200 to hold the active source 200 in place within the capsule 104. In some embodiments, the plug 108 can couple to the active source 200 via an adhesive. A receiver 120 can have a complementary shape to a distal end of the plug 108 to thereby receive and mate with the plug 108. The receiver 120 can connect to an interior wall of the capsule 102 via a weld joint. A proximal end of the receiver 120 can couple to a control wire 130 via a weld. The weld joint between the receiver and the capsule's interior wall can be removed, and the active source can then be removed from the capsule (e.g., by electric wire discharge) for re-activation and reuse. In further embodiments, the source can comprise a cap welded at each end for securing the active source within the capsule. Both welds can be removed, via, for example, electric wire discharge, and the source can be pushed out of the capsule from one end. Each of the capsule 104 and the control wire 130 can comprise stainless steel. The sample can be manipulated by an afterloader as is known in the art, such as, for example and without limitation, those provided by VARIAN BRAVOS, VARIAN GAMMAMEDPLUS, ELEKTA NUCLETRON MICROSLECTRON, or ECKERT & ZIEGLER BEBIG SAVINOVA.

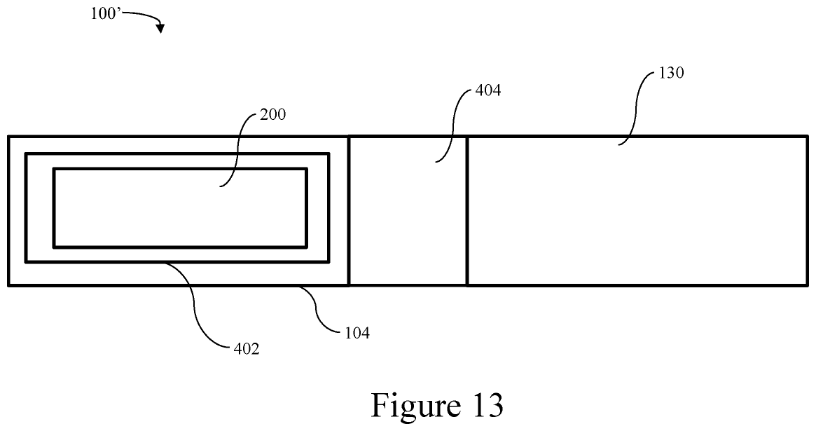

Referring to FIG. 13, a ¹⁶⁹Yb source 100′ can comprise an inner capsule 402 that can be disposed within the capsule 104. The active source 200 can be disposed within the inner capsule. The inner capsule 402 can be configured to be irradiated with active source 200 with neutrons during activation and reactivation of the active source 200. That is, the active source 200 and inner capsule 402 can be can be irradiated together as a single unit. The inner capsule 402 (and active source 200) can be removed from the capsule 104 during active source reactivation and then replaced into the same or a different capsule 104.

In some aspects, the capsule 104 can couple to the control wire 130 via a disposable segment 404 of material that can be cut to decouple the capsule 104 from the control wire 130. In some optional aspects, the disposable segment 404 can be a cylindrical metal segment. In some optional aspects, the disposable segment 404 can be the plug 108.

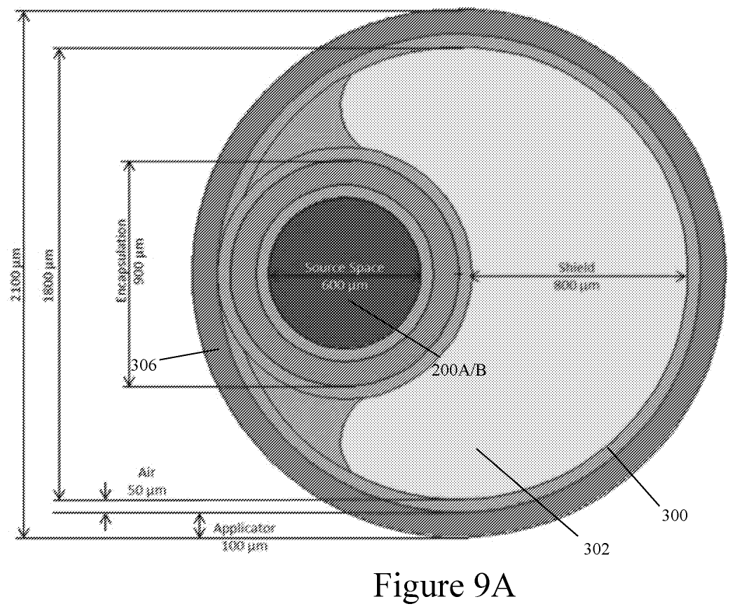

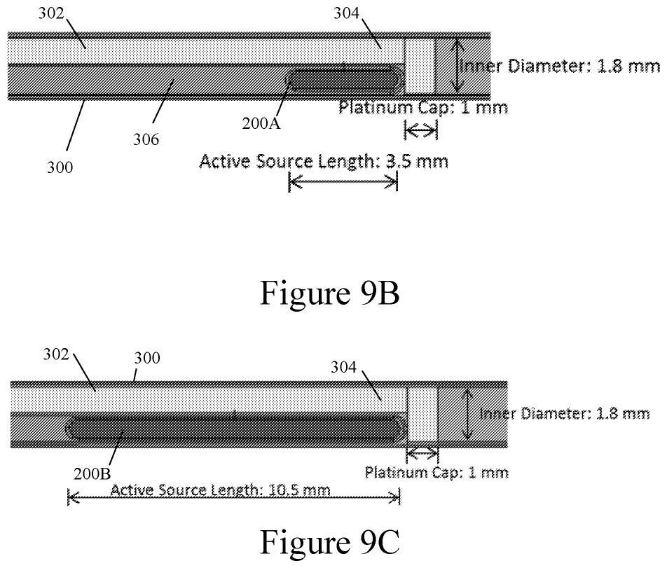

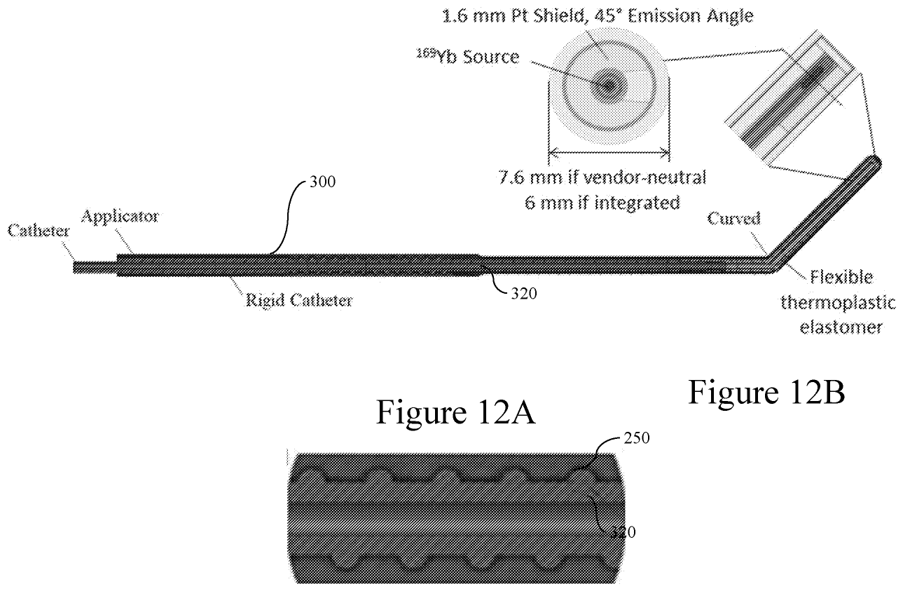

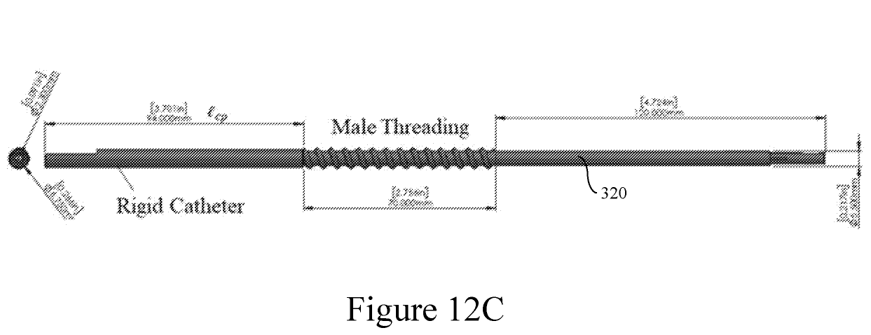

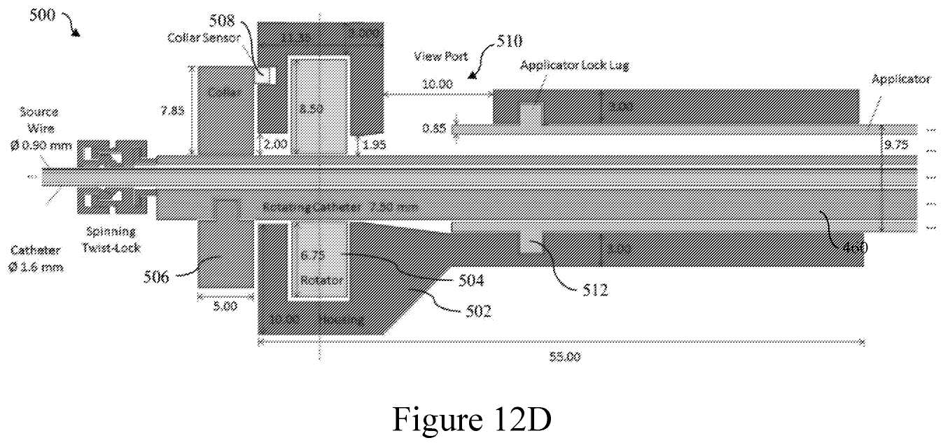

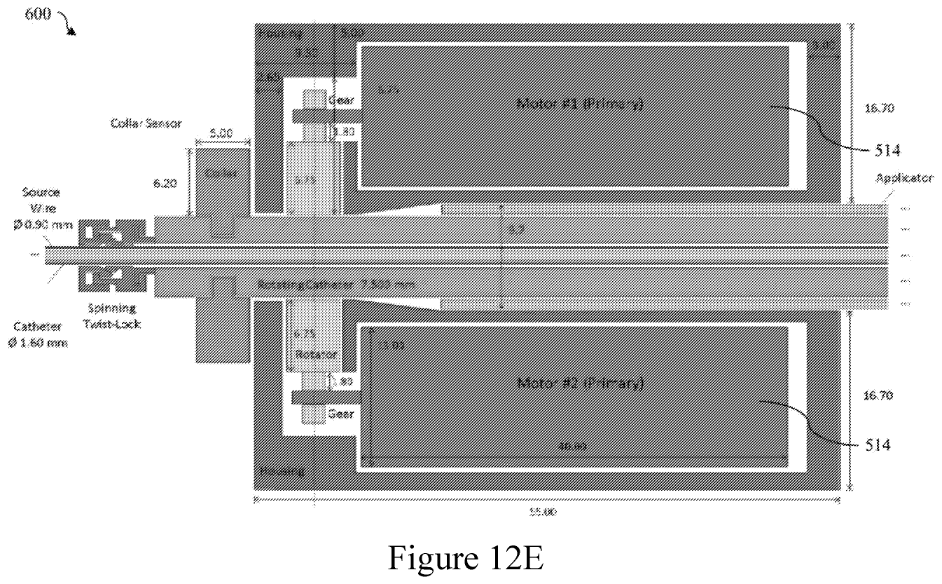

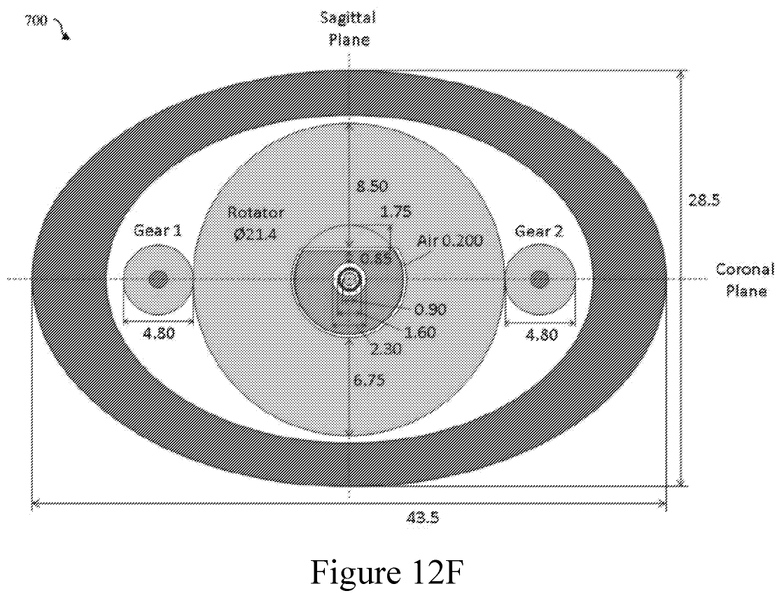

Referring to FIG. 12A-C, the ¹⁶⁹Yb source 100 can be used for rotating shield brachytherapy. According to one method, an applicator 250 (e.g., an intrauterine applicator) can be placed inside the patient. A rigid catheter 320 can then be inserted into the applicator 300. A motor drive system can be configured to rotate the rigid catheter 320. The catheter 320 can define an inner lumen that is configured to receive the ¹⁶⁹Yb source 100 as disclosed herein. The catheter 320 can optionally comprise a thermoplastic elastomer and can comprise a flexible end. Shields (e.g., shields 256 as in FIG. 9A) can be mounted to the end of the catheter 320. The shields 256 can optionally be disposed within the catheter 320. The applicator 250 can comprise female threads, and the catheter 320 can comprise male threads so that rotation of the catheter with respect to the applicator can cause longitudinal motion of the catheter 320 with respect to the applicator 256. Thus, when the active source is inserted into the catheter, rotation of the catheter can vary the longitudinal position of the radiation. Further, because of the shields, the rotation of the catheter can change the direction of emission of the radiation from the active source into the patient.

Prior to treatment, the rigid catheter can be rotated (e.g., using the motor drive system) until it reaches the distal end of the applicator. Treatment can begin when the remote afterloader drives the ¹⁶⁹Yb source 100, via the guidewire, to the distal end of the applicator, and the motor drive system begins to rotate. Throughout treatment the emission direction of the radiation shield can be controlled by the motor drive system.

FIGS. 12D-F illustrate a sagittal view of a motor drive system 500. The drive system 500 can be used with the applicator, catheter, and active source, as disclosed herein. In an aspect, the drive system 500 can have a housing 502, a rotator 504, a collar 506, a collar sensor 508 attached to the housing 502, a view port 510, an applicator lock lug 512. The rotator 504 can be coupled with a rotating catheter to rotate the catheter. The lock lug can affix the housing to the applicator. Motors 514 (FIG. 12E) can effect rotation of the rotators. The rotator can engage the catheter, for example, via frictional engagement or other means apparent to one skilled in the art, to rotate the catheter, thereby driving the catheter rotationally and longitudinally via the threaded engagement between the applicator and the catheter. Optionally, the drive system 500 can comprise two motors for the purposes of redundancy.

As the catheter rotates, it can travel longitudinally. The collar can indicate when the catheter is fully inserted into the applicator. That is, the collar can be positioned at a select distance from the end of the catheter, wherein the longitudinal position of the catheter when the collar reaches the collar sensor corresponds to the insertion distance at which the catheter extends to the distal end of the applicator (or to a select distance from the distal end of the applicator). When the catheter reaches the distal end of the applicator or the select distance from the distal end of the catheter, the collar can bias against the collar sensor, thereby causing the drive system to stop rotation once the catheter reaches a select longitudinal position. That is, the collar sensor can comprise a momentary switch that, when depressed, causes the motors to stop. For example, in one embodiment, the collar sensor can be a switch in communication with a controller (as further disclosed herein), wherein upon receiving a signal from the collar sensor, the controller can stop rotation of the motors. In this way, the collar and collar sensor can cooperate to limit the axial distance of travel of the catheter.

A spinning twist lock can couple the afterloader of the catheter to the rotating catheter so that the afterloader of the catheter can remain rationally stationary as the rotating catheter rotates.

FIG. 12E illustrates a coronal view of a drive system 600. In an aspect, the drive system 500 and the drive system 600 are different views of the same system. In an aspect, the drive system 900 can have a house, a rotator, a collar, and a first and a second motor. The first and second motor can be coupled to the rotator via a gear associated with each respective motor. The rotator can be coupled with a rotating catheter to rotate the catheter.

FIG. 12F illustrates an axial view of a drive system 700. In an aspect, the drive system 500, the drive system 600, and the drive system 700 are different views of the same system. In an aspect, the drive system 700 can have a first gear, a second gear, a rotator, and a housing.

Embodiments of the present disclosure can utilize two aspects: (i) minimizing expensive precursor waste by activating each source multiple times and (ii) minimizing the time the precursor needs to be in the nuclear reactor by maximizing the amount of ¹⁶⁸Yb in the active source volume, subject to the requirement that the dosimetric results cannot be compromised, nor can mechanical performance.

In one embodiment, the active source has a diameter of 0.6 mm and a length of 10.5 mm, with a total volume of approximately 3 mm³. Various other active source dimensions, and therefore, corresponding capsule 104 and control wire 130 dimensions are contemplated. For example, active source can have a length of 10.5 mm and a diameter of 0.69 mm to thereby provide a 4 mm³ active source. In yet another embodiment, the active source length can be less than 7.5 mm, 7.5 mm to 8 mm, 8 mm to 8.5 mm, 8.5 mm to 9 mm, 9 mm to 9.5 mm, 9.5 mm to 10 mm, 10 mm to 10.5 mm, or greater than 10.5 mm. The active source diameter can be less than 0.5 mm, 0.5 mm to 0.6 mm, 0.6 mm to 0.65 mm, 0.65 mm to 0.69 mm, 0.69 mm to 0.70 mm, or greater than 0.7 mm. The volume of the active element can be 2 cm³ and 2.5 cm³, 2.6 cm³ to 2.8 cm³, 2.8 cm³ to 3 cm³, 3 cm³ to 3.2 cm³, 3.2 cm³ to 3.5 cm³, 3.5 cm³ to 4 cm³, or greater than 4 cm³.

### D. ¹⁶⁹Yb Source Production and Use

¹⁶⁹Yb can be produced by irradiating the precursor in a research nuclear reactor, with or without the presence of existing ¹⁶⁹Yb within the active source. The precursor can be in many forms including a pellet, glass, or ceramic. A wide range of thermal neutron fluxes are available at various research reactors, and examples of maximum available thermal neutron fluxes are 6.0×10¹³ n cm⁻² s⁻¹ at the Massachusetts Institute of Technology Research Reactor (MITR-II), 6.0×10¹⁴ n cm⁻² s⁻¹ at the University of Missouri Research Reactor (MURR), and 5×10¹⁵ n cm⁻² s⁻¹ for the SM-3 reactor at the State Scientific Center—Research Institute of Atomic Reactors (RIAR) in Dimitrovgrad, Russia. A default average thermal neutron flux within the active source of 1×10¹⁴ n cm⁻² s⁻¹, which is realistically obtainable at multiple research reactors, including MURR, is used in activation calculations throughout this disclosure.

With the goal of delivering 27 Ci of ¹⁶⁹Yb to clinics following source activation, the time needed to allow undesirable radioactive impurities to decay away, to transport the radiation source wire to the receiving clinic, install it in the respective afterloader, and complete the associated quality assurance, is assumed to be 5 calendar days. Shorter or longer times can be used, depending on the process. For the 5-day assumption, the ¹⁶⁹Yb activity at the end of activation in the reactor can be 30 Ci to allow for the delivery of 27 Ci of ¹⁶⁹Yb to a clinic. In further aspects, the ¹⁶⁹Yb activity at the end of activation in the reactor can be selected to be greater or lower than 30 Ci, depending on the time required to provide the active source to the clinic, and the desired ¹⁶⁹Yb activity to be received at the clinic. For example, in further optional aspects, the ¹⁶⁹Yb activity at the end of activation can be about 31 Ci, about 32 Ci, about 33 Ci, about 35 Ci, about 38 Ci, or up to 40 Ci. In still further optional aspects, the ¹⁶⁹Yb activity at the end of activation can be about 29 Ci, or about 28 Ci, or about 27 Ci, about 26 Ci, or about 25 Ci.

As further explained below, it is contemplated that a 27 Ci ¹⁶⁹Yb HDR-BT source can produce the same dose rate in water at 1 cm from the source as a 10 Ci ¹⁹²Ir HDR-BT source. A 10 Ci (370 GBq) ¹⁹²Ir VARIAN VARISOURCE sample can have an air kerma strength (Sₖ) of 10.28×10⁻⁸ U/Bq (1 U=1 cGy cm² h⁻¹), a dose rate constant 1.101 cGy h⁻¹ U⁻¹, and therefore a dose rate 1 cm lateral to the source in water of 4.184×10⁴ cGy h⁻¹. To obtain the dose rate per unit activity in water at 1 cm from a ¹⁶⁹Yb source, the kerma strength per unit activity (Sₖ/A) can be obtained for such sources from two sources: a first experimental source provides a Sₖ/A of 0.042 μGy m² MBq⁻¹ hr⁻¹, in which is equal to 1.554 cGy cm² h⁻¹ mCi⁻¹ [U mCi⁻¹]; a second experimental source provides a Sₖ/A of 1.15 cGy cm² h⁻¹ mCi⁻¹ [U mCi⁻¹]. The reported dose rate constant for ¹⁶⁹Yb sources of 1.19 cGy h⁻¹ U⁻¹, thus the dose rate per activity value for the ¹⁶⁹Yb source can be experimentally found to be 1.84 cGy h⁻¹ mCi⁻¹ and 1.37 cGy h⁻¹ mCi⁻¹ via various methods. The average of these numbers is 1.605 cGy h⁻¹ mCi⁻¹, and, dividing that number by the dose rate in water at 1 cm from the 10 Ci ¹⁹²Ir source, it can be found that a 26 Ci ¹⁶⁹Yb source is needed to match the 10 Ci ¹⁹²Ir dose rate. Adding a 4% safety margin, the 27 Ci quantity for the ¹⁶⁹Yb activity required to match the dose rate at 1 cm in water for a 10 Ci ¹⁹²Ir HDR BT source can be determined.

The minimum useful activity of a clinical ¹⁶⁹Yb radiation source is highly dependent upon the brachytherapy application, as LDR-BT, PDR-BT, or MDR-BT procedures could be performed with lower ¹⁶⁹Yb activities than needed for HDR-BT. It can be conservatively assumed that the minimum useful clinical dose rate is that which provides that same dose rate in water at 1 cm lateral to the source as a ¹⁹²Ir source used for HDR-BT just prior to a typical source change. Such an ¹⁹²Ir source typically has an initial activity of 10 Ci and is replaced after 90 days. With a half-life of 73.83 days, the ¹⁹²Ir activity at the time of replacement under these assumptions would be 4.30 Ci. The ¹⁶⁹Yb activity that matches the dose rate of a ¹⁹²Ir at the time of replacement is thus (27 Ci) (4.3/10)=11.6 Ci, which, for an initial clinical ¹⁶⁹Yb activity of 27 Ci, occurs after 39 days of clinical use.

### E. Radioactive Impurities

The primary radioactive impurities of concern in a ¹⁶⁹Yb source are ¹⁷⁵Yb and ¹⁷⁷Yb, which have half-lives of 105 h and 1.9 h, respectively. The radioactive impurities are produced during thermal neutron activation of the precursor due to the presence of isotopic impurities of ¹⁷⁴Yb and ¹⁷⁶Yb. The expected ¹⁷⁵Yb activity following 12 days of neutron irradiation of 17.1% enriched ¹⁶⁸Yb—Yb₂O₃ at 1×10¹⁴ n cm⁻² s⁻¹ is 0.2% of the ¹⁶⁹Yb activity, and 10 days of cooling are recommended prior for decay of radionuclidic impurities. In one exemplary test, the ratio of ¹⁶⁸Yb to ¹⁷⁴Yb in the initial sample was 17.1/15.5=1.10. With 82% enriched ¹⁶⁸Yb—Yb₂O₃ precursor, approximate ¹⁷⁴Yb and ¹⁷⁶Yb abundances are 3% and 1% (Trace Sciences International), respectively, and the resulting ¹⁷⁵Yb impurity following activation would be (0.2%) (1.10) (3/82)=0.008%. This is a ¹⁷⁵Yb-to-¹⁶⁹Yb activity reduction by a factor of 1/25 relative to that of the 17.1% enriched ¹⁶⁸Yb—Yb₂O₃ precursor, which is equivalent to 20 days of cooling given the 105 h (4.38 d) half-life of ¹⁷⁵Yb. This is effectively 10 days beyond the IAEA recommended 10 days of cooling for the ¹⁶⁹Yb source with 0.2% activity ¹⁷⁵Yb impurity. In the exemplary test, the activity of the lower half-life isotope, ¹⁷⁵Yb, was considered acceptable after 10 days of cooling when the ratio of ¹⁷⁴Yb to ¹⁷⁶Yb in the initial sample was 15.5%/4.5%=3.4. As the ratio of ¹⁷⁴Yb to ¹⁷⁶Yb in the 82%-enriched ¹⁶⁸Yb—Yb₂O₃ precursor is 3—lower than that from the cited study—the produced ¹⁷⁵Yb from the 82%-enriched precursor would be sufficiently low immediately after activation to be acceptable without additional cooling. Therefore, the reduction in ¹⁷⁵Yb obtained by using 82% or higher enriched precursor is sufficient to enable immediate shipment of the ¹⁶⁹Yb source following activation, thus a 5-day period between removal of the ¹⁶⁹Yb source from the nuclear reactor and installation in a clinical afterloader is realistic.

### F. ¹⁶⁹Yb Activation Equations

Precursor quantity (source volume), nuclear reactor time, and source activity are quantitatively related, as shown with respect to the equations for radioactive source activation. As further explained below, the amount of ¹⁶⁸Yb precursor material and nuclear reactor time needed to produce one year's worth of ¹⁶⁹Yb for a single clinical afterloader, can be calculated as a function of active source volume.

Three isotopes of a given element, indexed by 1, 2, and 3, can be considered. Isotope 1 is stable, has mass number M-1, and becomes the active source isotope after absorbing a thermal neutron. Examples of Isotope 1 are ¹⁹¹Ir and ¹⁶⁸Yb, and their relevant physical properties are listed in Table 1. Isotope 2 is the gamma ray emitting (therapeutic) active source isotope with mass number M, a half-life of t_{1/2} s, a decay constant of λ₂=ln(2)/t_{1/2} s⁻¹, and is the result of thermal neutron absorption by isotope 1 with cross section τ₁ cm². Examples of Isotope 2 are ¹⁹²Ir and ¹⁶⁹Yb. Isotope 3 is stable, has mass number M +1, and is the result of thermal neutron absorption by isotope 2 with cross section τ₂. Examples of isotope 3 are ¹⁹³Ir and ¹⁷⁰Yb. Given an average thermal neutron flux within the active source of φ n cm⁻² s⁻¹, including the effects of thermal neutron attenuation, changes with time, t, of the numbers of the atoms, Nₘ(t) of each isotope, m, (m=1, . . . , 3), for a given element can be described by the following three differential equations:

$$\begin{matrix} \frac{d {N}_{1} ( t )}{d t} = - {φσ}_{1} {N}_{1} ( t ) , & ( 1 ) \\ \frac{d {N}_{2} ( t )}{d t} = {φσ}_{1} {N}_{1} ( t ) - {λ}_{2} {N}_{2} ( t ) - {φσ}_{2} {N}_{2} ( t ) = {φσ}_{1} {N}_{1} ( t ) - ( {λ}_{2} + {φσ}_{2} ) {N}_{2} ( t ) , \\ & ( 2 ) \\ and & \\ \frac{d {N}_{3} ( t )}{d t} = {φσ}_{2} {N}_{2} ( t ) , & ( 3 )\end{matrix}$$

with the boundary conditions:

N₁(0)=N₁⁰, N₂(0)=N₂⁰, and N₃(0)=N₃⁰, (4)

which are the respective isotope counts at time t=0. Equations (1), (2), and (3) can be solved by taking the Laplace transform of the variables, yielding the following equations for the isotopic quantities:

$$\begin{matrix} {N}_{1} ( t ) = {N}_{1}^{0} {e}^{- {φσ}_{1} t} , & ( 5 ) \\ {N}_{2} ( t ) = \frac{{φσ}_{1}}{{φσ}_{2} + {λ}_{2} - {φσ}_{1}} {N}_{1}^{0} [ {e}^{- {φσ}_{1} t} - {e}^{- ( {φσ}_{2} + {λ}_{2} ) t} ] + {N}_{2}^{0} {e}^{- ( {φσ}_{2} + {λ}_{2} ) t} , & ( 6 ) \\ and & \\ {N}_{3} ( t ) = \frac{{φσ}_{2}}{{φσ}_{3} - {φσ}_{2}} {N}_{2}^{0} ( {e}^{- {φσ}_{2} t} - {e}^{- {φσ}_{3} t} ) + {N}_{3}^{0} {e}^{- {φσ}_{3} t} . & ( 7 )\end{matrix}$$

For the case of N₂⁰=0, which occurs when there is no initial therapeutic isotope (¹⁹²Ir or ¹⁶⁹Yb) activity, Equation (6) reduces to the form of Equation (32). Isotope 3 can be capable of undergoing further thermal neutron absorptions, and Equation (7) accounts for isotope 3 (¹⁷⁰Yb or ¹⁹³Ir) generation and loss. By the definition of radioactivity, the activity of isotope m at time t can be calculated as:

Aₘ(t)=λₘNₘ(t), (8)

where λₘ is the decay constant for isotope m.

Equation (6) can be differentiated with respect to time, set equal to zero, and solved for tₛ, the time after the start of irradiation at which saturation (the maximum activity) of isotope 2 (¹⁹²Ir or ¹⁶⁹Yb) occurs. The result is the following:

$${t}_{s} = - \frac{1}{{φσ}_{2} + {λ}_{2} - {φσ}_{1}} ln [ \frac{{φσ}_{1}}{{φσ}_{2} + {λ}_{2}} \cdot \frac{{φσ}_{1} {N}_{1}^{0}}{{φσ}_{1} {N}_{1}^{0} - ( {φσ}_{2} + {λ}_{2} - {φσ}_{1} ) {N}_{2}^{0}} ] . \qquad ( 9 )$$

The activity of isotope 2 at saturation can be calculated as:

A_{2,s}=λ₂N₂(tₛ), (10)

and a general expression for the number of therapeutic isotopes (¹⁹²Ir or ¹⁶⁹Yb) in the source at the time of saturation is:

$${N}_{2} ( {t}_{s} ) = {\frac{{φσ}_{1} {N}_{1}^{0}}{{φσ}_{2} + {λ}_{2} - {φσ}_{1}} [ \frac{{φσ}_{1}}{{φσ}_{2} + {λ}_{2}} \cdot \frac{{φσ}_{1} {N}_{1}^{0}}{{φσ}_{1} {N}_{1}^{0} - ( {φσ}_{2} + {λ}_{2} - {φσ}_{1} ) {N}_{2}^{0}} ]}^{\frac{{φσ}_{1}}{{φσ}_{2} + {λ}_{2} - {φσ}_{1}}} + ( {N}_{2}^{0} - \frac{{φσ}_{1} {N}_{1}^{0}}{{φσ}_{2} + {λ}_{2} - {φσ}_{1}} ) \times {[ \frac{{φσ}_{1}}{{φσ}_{2} + {λ}_{2}} \cdot \frac{{φσ}_{1} {N}_{1}^{0}}{{φσ}_{1} {N}_{1}^{0} - ( {φσ}_{2} + {λ}_{2} - {φσ}_{1} ) {N}_{2}^{0}} ]}^{\frac{{φσ}_{2} + {λ}_{2}}{{φσ}_{2} + {λ}_{2} - {φσ}_{1}}} \qquad ( 11 )$$

For the case of N₂⁰=0, Equation (11) reduces to:

$${N}_{2} ( {t}_{s} ) = \frac{{φσ}_{1}}{{φσ}_{2} + {λ}_{2} - {φσ}_{1}} {N}_{1}^{0} [ {( \frac{{φσ}_{1}}{{φσ}_{2} + {λ}_{2}} )}^{\frac{{φσ}_{1}}{{φσ}_{2} + {λ}_{2} - {φσ}_{1}}} - {( \frac{{φσ}_{1}}{{φσ}_{2} + {λ}_{2}} )}^{\frac{{φσ}_{2} + {λ}_{2}}{{φσ}_{2} + {λ}_{2} - {φσ}_{1}}} ] . \qquad ( 12 )$$

### G. Exemplary Model Benchmarking

The model for source activation was benchmarked against published literature for ¹⁹²Ir activation to assess its accuracy and determine if it can be applied confidently for modeling ¹⁶⁹Yb generation. Results are shown in Table 2Table, which indicates that the calculated model was accurate to within 3.3% of reference values from two previous works. The model is thus sufficiently accurate for ¹⁹²Ir activation calculations to justify its application to ¹⁶⁹Yb production.

TABLE 2
Benchmark results for activation calculations, which were
performed for ¹⁹²Ir, since references exist. Definitions: φ = average
thermal neutron flux inside active source; t = time in reactor; SA =
specific activity at time t; Ref. = reference citation or quantity; Calc. =
calculated value; Diff. = percentage difference between Calc.
and Ref., Sat. = saturation.

| $φ [ \frac{n}{{cm}^{2} s} ]$ | t [d] Ref. Calc. Diff. | | | $\begin{matrix}SA [ \frac{Ci}{g Ir} ] , {[ \frac{Ci}{{g}^{191} Ir} ]}^{*} \\ Ref . Calc . Diff .\end{matrix}$ | | |
|---|---|---|---|---|---|---|
| 1 × 10¹² | 7 | | 0 | 1.85 | 1.91 | 3.2% |
| 1 × 10¹² | 28 | | 0 | 6.73 | 6.94 | 3.1% |
| 1 × 10¹² | 504 (Sat.) | | 0 | 28.9 | 28.5 | −1.4% |
| 1 × 10¹³ | 258 | 261 | 1% | 579* | 580* | 0.2% |
| 2 × 10¹⁴ | 42.8 | 41.4 | −3.3% | 2,250* | 2,274* | 1% |

### H. Thermal Neutron Attenuation Within the Active Source Volume

An active source volume containing precursor material and some existing quantity of the therapeutic isotope can attenuate the thermal neutrons impinging upon it. Thus, the thermal neutron flux at the center of the active source can be lower than that at the surface. This attenuation can be implicitly accounted for in the calculations above since the quantity, φ, is the average thermal neutron flux inside the active radiation source, thus including the effects of neutron attenuation in the source. The thermal neutron flux that would need to be delivered in the volume in which the active source will be placed within the reactor, i.e., the advertised reactor neutron flux, φ₀, would thus need to be greater than the average thermal neutron flux inside the active radiation source, φ.

Gaining a thorough understanding of the distribution of thermal neutrons within an active radiation source placed inside a research reactor, and the change in the distribution over time as isotopes are converted, could be accomplish using Monte Carlo simulations. Here, an analytical thermal neutron attenuation calculation demonstrates that Yb precursor material is substantially less attenuating than Ir precursor material of the same geometric size. The thermal neutron flux at depth, [cm], can be calculated as:

φ(x)=φ₀e^{-ux}, (13)

where φ₀ is the flux of the thermal neutron beam at the surface of the active source and the attenuation coefficient, μ, [cm⁻¹] is obtained as:

$$μ = \frac{σ {N}_{A} ρ}{{m}_{a}} , \qquad ( 14 )$$

where N_{A} is Avogadro's number (6.023×10²³ g/mol), ρ is the density of the medium [g/cm³], mₐ is the atomic mass of the medium [g/mol], τ is the thermal neutron absorption cross section for the active source [cm²], which is a linear combination of τ₁, τ₂, and τ₃, weighted by the relative abundances of isotopes 1 (¹⁹¹Ir, ¹⁶⁸Yb), 2 (¹⁹²Ir, ¹⁶⁹Yb), and 3 (¹⁹³Ir, ¹⁷⁰Yb) from Table 1. In this analysis a will be set equal to τ₁, corresponding to the case of an active source that is pure un-activated precursor material. Attenuation coefficients for Ir and Yb precursor materials can be calculated using ρ-values of 22.56 g/cm³ and 8.15 g/cm³, respectively, and mₐ-values of 192.2 and 173, respectively, and are 88.1 cm⁻¹ and 53 cm⁻¹, respectively. Assuming the precursor material has a diameter of 0.6 mm, the distance on an axial cross section between the surface of the active source and the center is 0.3 mm, and the thermal neutron transmission between those points for Ir and Yb active sources would be 7% and 20%, respectively. These relative attenuation values will shift throughout the activation process as the respective quantities of ¹⁶⁸Yb and ¹⁶⁹Yb change with time.

Based on these attenuation calculations, it can be understood that, if the geometric and physical properties of Ir and Yb active source precursors are set such that the total thermal neutron irradiation time needed to generate a one-year supply of ¹⁹²Ir and ¹⁶⁹Yb are equal according to activation model of Equation (5), Equation (6), and Equation (7), then, once thermal neutron attenuation is fully accounted for, the total actual neutron irradiation time needed to generate ¹⁶⁹Yb can be less than for ¹⁹²Ir.

### I. Efficient Activation

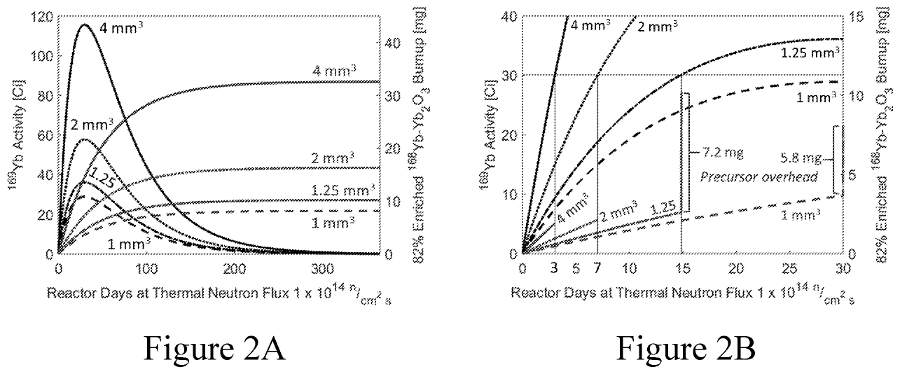

Activation of the precursor in a nuclear reactor at a flux of 1×10¹⁴ n cm⁻² s⁻¹ can have an estimated cost of $1,500 per week, and the rate at which the activity of the therapeutic isotope, ¹⁶⁹Yb, increases in a reactor can have an impact on the price of the resulting radiation source. This concept is illustrated in FIGS. 2A and 2B, in which the activation curves and precursor burnup curves are shown for precursor volumes of 1, 1.25, 2, and 4 mm³. FIGS. 2A and 2B illustrate, for one year of continuous activation and thirty days of continuous activation, respectively, simulated activation and precursor burnup curves for four volumes of 82% enriched of ¹⁶⁸Yb—Yb₂O₃ ranging from 1 mm³ to 4 mm³, with a density of 8.15 g/cm³, in a nuclear reactor with an average thermal neutron flux within the active source of 1×10¹⁴ n cm⁻² s⁻¹. In FIG. 2A the activation and precursor burnup curves are shown for a full year of precursor irradiation by thermal neutrons at an average thermal neutron flux within the active source of 1×10¹⁴ n cm⁻² s⁻¹, indicating that activity saturation occurs at approximately 30 days after the start of activation. Following one year of irradiation by thermal neutrons, nearly all ¹⁶⁸Yb atoms in the precursor have been activated regardless of the active source volume, corresponding to an activation of the following precursor (¹⁶⁸Yb—Yb₂O₃) masses: 8.15, 10.19, 16.30, and 32.60 mg for 1, 1.25, 2, and 4 mm³ precursor volumes, respectively. Focusing on the first 30 days in the activation process, FIG. 2B indicates the activation times needed to achieve the goal ¹⁶⁹Yb activity at the end of activation of 30 Ci.

Two primary principles can dictate activation efficiency: the quantity of ¹⁶⁸Yb isotopes in the active source and activation linearity. At the start of the activation process, assuming no ¹⁶⁹Yb is already present in the active source, N₁(t)>>N₂(t) (¹⁶⁸Yb isotopes>>¹⁶⁹Yb isotopes), and, as indicated by Equation (2), the rate of change in the number of ¹⁶⁹Yb isotopes, dN₂(t)/dt, is nearly a linear function of N₁(t) (¹⁶⁸Yb isotope count) since the first term on the right hand side dominates. This means the time efficiency of the activation process can increase with the number of ¹⁶⁸Yb atoms present, as there are more targets that absorb thermal neutrons inside the active source. As the ¹⁶⁸Yb in the active source is converted to ¹⁶⁹Yb, the number of ¹⁶⁸Yb isotopes (targets) diminishes as described by Equation (5), reducing the rate of ¹⁶⁹Yb generation and therefore activation efficiency. The activation curve can still remain approximately linear if sufficient ¹⁶⁸Yb remains in the active source, as shown for the 2 mm³ and 4 mm³ sources in FIG. 2B, indicating activation times of 3-7 days. The activation curve can become asymptotic as the quantity of remaining ¹⁶⁸Yb approaches the minimum required to generate a 30 Ci source, as shown in FIG. 2B for the 1 mm³ and 1.25 mm³ sources. This is because ¹⁶⁹Yb generation is being limited by dwindling ¹⁶⁸Yb availability, and is also competing with ¹⁶⁹Yb losses due to radioactive decay and thermal neutron absorption by the ¹⁶⁹Yb isotopes. The asymptotic activation effect substantially decreases activation efficiency below that for linear activation, with 15-30 day activation times for the 1 mm³ and 1.25 mm³ sources, and, for the case of the 1 mm³ active source, the goal activity of 30 Ci theoretically is never reached.

### J. Precursor Overhead

During the ¹⁶⁹Yb generation process described by Equation (6), some ¹⁶⁸Yb is converted to ¹⁶⁹Yb by thermal neutron absorption with a rate constant of φτ₁ [s⁻¹], some ¹⁶⁹Yb is lost by radioactive decay to ¹⁶⁹Tm with a 32 day half-life and a rate constant of λ₂ [s⁻¹], and some ¹⁶⁹Yb absorbs thermal neutrons to become ¹⁷⁰Yb at a rate constant of φτ₂ [s⁻¹]. These processes must be accounted for to determine the minimum amount of ¹⁶⁸Yb needed to reach the threshold clinical ¹⁶⁹Yb activity. Using Equation (12), the precursor overhead needed to generate a ¹⁶⁹Yb source can be calculated for an idealized situation of an infinite neutron flux, and also for the realistic situation in which the default average thermal neutron flux within the active source of 1×10¹⁴ n cm⁻² s⁻¹.

The mass of ¹⁶⁸Yb needed to produce a ¹⁶⁹Yb source with an activity of 30 Ci for the idealized case of an infinite thermal neutron flux can be calculated. This situation can be assumed to occur at the time of saturation, tₛ, which means the exact amount of ¹⁶⁸Yb would be irradiated by thermal neutrons that would reach 30 Ci of ¹⁶⁹Yb activity in the instant before the overall ¹⁶⁹Yb activity starts to decrease due to burnup up the initial ¹⁶⁸Yb in the neutron environment and conversion of ¹⁶⁹Yb to ¹⁷⁰Yb. Equation (11) can be simplified to determine this by taking the limit as cp becomes infinite, obtaining an expression relating N₂^{min}, the minimum number of ¹⁶⁹Yb atoms needed to obtain the desired activity, to N₁^{min}, the minimum number of ¹⁶⁸Yb atoms needed to generate that activity in a reactor with an infinite neutron flux:

$${N}_{2}^{min} = {N}_{2} ( {t}_{s} ) = {N}_{1}^{min} {\frac{{σ}_{1}}{{σ}_{2} - {σ}_{1}} [ \frac{{σ}_{1}}{{σ}_{2}} \cdot \frac{{σ}_{1} {N}_{1}^{0}}{{σ}_{1} {N}_{1}^{0} - ( {σ}_{2} - {σ}_{1} ) {N}_{2}^{0}} ]}^{\frac{{σ}_{1}}{{σ}_{2} - {σ}_{1}}} + {( {N}_{2}^{0} - \frac{{σ}_{1} {N}_{1}^{0}}{{σ}_{2} - {σ}_{1}} ) [ \frac{{σ}_{1}}{{σ}_{2}} \cdot \frac{{σ}_{1} {N}_{1}^{0}}{{σ}_{1} {N}_{1}^{0} - ( {σ}_{2} - {σ}_{1} ) {N}_{2}^{0}} ]}^{\frac{{σ}_{2}}{{σ}_{2} + {σ}_{1}}} . \qquad ( 151 )$$

Equation (15) can be simplified to the following for the case of an active source that does not contain ¹⁶⁹Yb, thus N₂⁰=0:

$${N}_{2}^{min} = {N}_{2} ( {t}_{s} ) = {N}_{1}^{min} \frac{{σ}_{1}}{{σ}_{2} - {σ}_{1}} [ {( \frac{{σ}_{1}}{{σ}_{2}} )}^{\frac{{σ}_{1}}{{σ}_{2} - {σ}_{1}}} - {( \frac{{σ}_{1}}{{σ}_{2}} )}^{\frac{{σ}_{2}}{{σ}_{2} - {σ}_{1}}} ] . \qquad ( 162 )$$

Using parameters from Table 1, one obtains N₂^{min}=N₁^{min}·0.29 from Equation (16), therefore A₂^{min}=λ₂N₂^{min}=λ₂N₁^{min}·0.29 and

$${N}_{1}^{min} = \frac{{A}_{2}^{min}}{0.29 \cdot {λ}_{2}} = \frac{30 Ci \cdot 3.7 \times 1 {0}^{10} \text{Bq/Ci}}{7.25 \times 1 {0}^{- 8} {s}^{- 1}} = 1.54 \times 1 {{0}^{19} }^{168} Yb atoms . \qquad ( 17 )$$

This corresponds to the following precursor mass:

$$1 .54 \times 1 {0}^{19 \; 168} Yb atoms \frac{1 Yb atom}{{0.82 }^{168} Yb atoms} \frac{173 g \text{Yb/mol}}{6.02 \times 1 {0}^{23} \text{atoms/mol}} \frac{394 g {Yb}_{2} {O}_{3}}{346 g {Yb}_{2}} = 6.1 {mg }^{168} Yb - {Yb}_{2} {O}_{3} \qquad ( 3 )$$

Thus the minimum amount of 82% enriched ¹⁶⁸Yb—Yb₂O₃ precursor needed to obtain an activity of 30 Ci of ¹⁶⁹Yb is 6.1 mg, even when using a reactor with an infinite neutron flux. With a precursor density of 8.15 mg/mm³, this corresponds to 0.76 mm³ of active source volume, which would cost $4,221 per physical source.

Because there are no thermal neutron sources with infinite flux, the precursor overhead can also be calculated for the default average thermal neutron flux within the active source of 1×10¹⁴ n cm⁻² s⁻¹. Equation (12) can be used to obtain the relationship N₂^{min}=N₁^{min}·0.21, resulting in a minimum ¹⁶⁸Yb atomic quantity of:

$${N}_{1}^{min} = \frac{{A}_{2}^{min}}{0.21 \cdot {λ}_{2}} = \frac{30 Ci \cdot 3.7 \times 1 {0}^{10} \text{Bq} \text{/} \text{Ci}}{5.25 \times 1 {0}^{- 8} {s}^{- 1}} = 2.1 \; 3 \times 1 {{0}^{19} }^{168} Yb atoms , \qquad ( 4 )$$

corresponding to a precursor mass of:

$$2 .13 \times 1 {0}^{19 \; 168} Yb atoms \frac{1 Yb atom}{0.8 {2}^{ 168} Yb atoms} \frac{173 g \text{Yb/mol}}{6.02 \times 1 {0}^{23} \text{atoms/mol}} \frac{394 g {Yb}_{2} {O}_{3}}{346 g {Yb}_{2}} = 8.48 {mg }^{168} Yb - {Yb}_{2} {O}_{3} . \qquad ( 5 )$$

For a Yb₂O₃ density of 8.15 mg/mm³, this corresponds to 1.04 mm³ of active source volume, which would cost $5,868 per physical source. Thus, for this example, reducing the flux from an infinite neutron flux to an average thermal neutron flux within the active source of 1×10¹⁴ n cm⁻² s⁻¹ only reduces precursor overhead cost by $1,647, or 28%.

Precursor overhead can also be calculated for the case when a ¹⁶⁹Yb source is re-inserted into a reactor for reactivation 5 days after reaching its assumed minimum clinical activity of 11.6 Ci, thus the ¹⁶⁹Yb activity upon re-insertion into the reactor would be 10.4 Ci. The minimum precursor mass of 82% enriched ¹⁶⁸Yb—Yb₂O₃ needed to reach an activity of 30 Ci is 7.8 mg, which can be obtained by numerically solving Equation (11) for N₁⁰=N₁^{min} for an N₂⁰ of 10.4 Ci/λ₂.

### K. Re-Activation

As shown in FIG. 2A, the 2 mm³ and 4 mm³ sources have clearly not been activated to their full potential after reaching 30 Ci, and substantial useful precursor remains in the active source volume. These sources can be re-activated after clinical usage. However, re-activation of the 1 mm³ and 1.25 mm³ sources to 30 Ci would not be feasible since the remaining precursor after the first activation, 5.8 mg and 7.2 mg, respectively, is below the required precursor overhead for re-activation of 7.8 mg in each case.

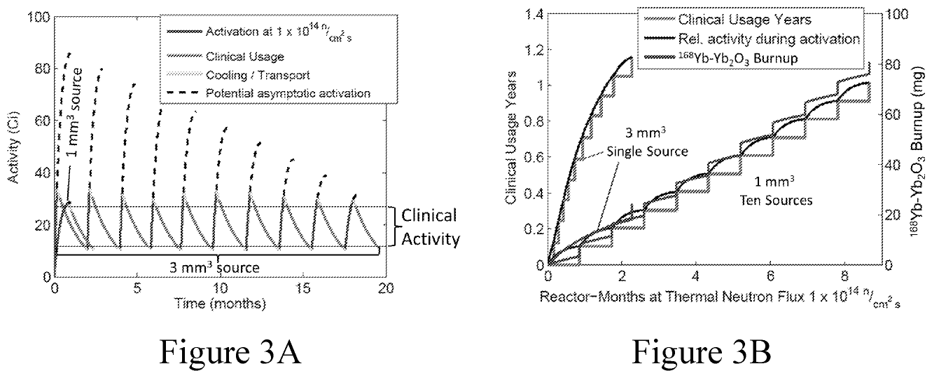

Activation and re-activation sequences can be simulated for 1 mm³ and 3 mm³ sources using Equation (6), and the resulting activation curves are shown in FIG. 3A, along with plots in FIG. 3B indicating clinical usage years and precursor burnup with reactor months at an average thermal neutron flux within the active source of 1×10¹⁴ n cm⁻² s⁻¹. FIGS. 3A and 3B illustrate that 80 mg/yr versus 21 mg/yr of precursor per clinical year is needed for 1 mm³ versus 3 mm^{3 169}Yb sources, which indicates a significant financial advantage for the larger sources, at $14,532 versus $55,360, respectively. More specifically, FIG. 3A illustrates activity vs. time curves for ¹⁶⁹Yb sources with active source volumes of 1 mm³ and 3 mm³. A 1 mm³ source can be activated once before exhausting its ¹⁶⁸Yb precursor supply below the threshold needed for effective re-activation (7.8 mg in this example), and it will never meet the target activity of 30 Ci of ¹⁶⁹Yb (as illustrated in FIG. 2B). A 3 mm³ source, however, can be activated and re-activated at total of 10 times. FIG. 3B illustrates Clinical usage years obtained and precursor burnup with reactor-months assuming an average thermal neutron flux within the active source of 1×10¹⁴ n cm⁻² s⁻¹, for 1 mm³ and 3 mm³ active sources. For 1 mm³ sources, 256 reactor-days and 80 mg per year of 82%-enriched ¹⁶⁸Yb—Yb₂O₃ precursor can be needed to generate a one-year clinical supply of ¹⁶⁹Yb. For 3 mm³ sources, only 59 reactor-days and 21 mg of precursor can be needed. This advantage is a result of minimizing wasted precursor (as illustrated in FIG. 2B) by using the re-activation strategy. Reactor-days are reduced from 256 to 59 by using a 3 mm³ versus 1 mm³ source since three times more ¹⁶⁸Yb atoms are initially present to be activated in the 3 mm³ volume, and also since activation of the 3 mm³ source is a predominantly linear process, whereas the activation process for the 1 mm³ source is asymptotic (FIG. 2B). This represents a total reduction in ¹⁶⁹Yb annual precursor and reactor time costs of 74% and 77%, respectively.

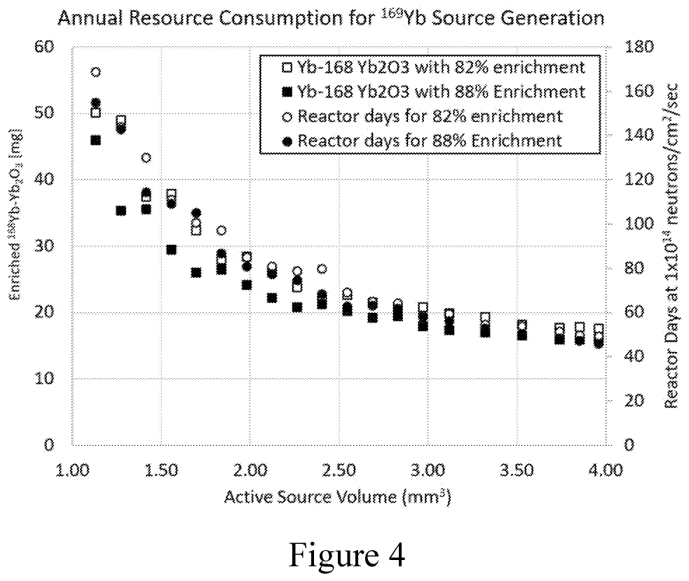

To quantify the relative costs required to generate ¹⁶⁹Yb sources with the claimed active volumes of 2-4 mm³, calculations can be performed to determine the number of reactor days at an average thermal neutron flux within the active source of 1×10¹⁴ n cm⁻² s⁻¹ and precursor mass needed to generate a year's clinical supply of ¹⁶⁹Yb over a 1-4 mm³ range of active source volumes. The calculated result are shown in FIG. 4, which illustrates resource consumption needed for one year of clinical ¹⁶⁹Yb source generation. Data shown were generated assuming the thermal neutron flux at the surface of the active radiation source during activation is great enough so the average thermal neutron flux within the active source volume is 1×10¹⁴ n cm⁻² s⁻¹. Two precursor enrichment levels were considered for this illustration: 82% and 88%. Whether 82% enriched or 88% enriched ¹⁶⁸Yb—Yb₂O₃ is used to generate the ¹⁶⁹Yb sources, the precursor mass and the number of reactor days needed per year drop by 26-31% when the source volume increases from 2 mm³ to 3 mm³. The drop is another 11-21% between 3 mm³ and 4 mm³ source volumes at both enrichment levels.

### L. Impact of Source Volume: Dosimetric Considerations

The impact of source volume on the dosimetric results for cervical and prostate cancer were thoroughly evaluated in a pair of treatment planning studies. Both studies were done with Institutional Review Board (IRB) approval. The POGS optimization technique was used for treatment planning for the cervical cancer and prostate cancer cases.

For all treatment plans for which ¹⁶⁹Yb was considered, both 1 mm³ and 3 mm³ sources, which had active source diameters of 0.60 mm and lengths of 3.5 mm and 10.5 mm, respectively, were modeled using the Monte Carlo N-Particle Transport (MCNP) radiation transport code. The ¹⁹²Ir source was modeled using MCNP as a VARIAN VARISOURCE afterloader source, with active source dimensions of 0.34 mm diameter and 5 mm length. The same energy deposition tally geometry, based in spherical coordinates, was used for the ¹⁹²Ir and ¹⁶⁹Yb sources, and a sufficient number of particles were transported such that the dose-weighted relative error in all tally cells was 0.011 or less as described by Adams et al (2014). Source activity was set to 10 Ci for ¹⁹²Ir and 27 Ci for ¹⁶⁹Yb, to generate treatment plans with the shortest feasible delivery times, representing freshly-replaced clinical sources. The activities were reduced to 4.3 Ci for ¹⁹²Ir and 11.6 Ci for ¹⁶⁹Yb and delivery times for the corresponding activities were re-calculated, representing the longest clinical delivery times, which would be delivered just prior to replacing the radiation sources.

1. Cervical Cancer Dosimetric Considerations

For cervical cancer, 37 patients were considered, all with high-risk clinical target volumes (HR-CTVs) of 41 cm³ or greater (mean 79 cm³, standard deviation 36 cm³, minimum 41 cm³, maximum 192 cm³). As cervical cancer patients were found to benefit most from the IC/IS approach if their HR-CTV was 30 cm³ or larger in the RETROEMBRACE study, the patients considered represent the candidates for RSBT who would benefit most from the resulting improved dose conformity. Contours for HR-CTV, bladder, rectum, and sigmoid colon were generated by a radiation oncologist based on magnetic resonance imaging (MRI) datasets. Plans for IC/IS, IC, and RSBT using ¹⁹²Ir and ¹⁶⁹Yb and shield emission angles of 45°, 180°, and both 45° and 180° in the same rotating catheter, with the 180° shield distal to the 45° shield, were generated. The RSBT rotating catheter with the combination of the 45° and 180° shield emission angles was considered since using both emission angles may improve HR-CTV D₉₀ relative to rotating catheters that would use 45° and 180° shield emission angles alone, and is expected to decrease overall delivery times below those for the 45° shields alone. The Varian Medical Systems (Palo Alto, Calif.) BrachyVision treatment planning system was used to define the positions of the IC and IC/IS applicators for all patients, and the Varian Titanium Fletcher-Style IC/IS tandem and ovoid applicator with up to four interstitial needles for each ovoid, up eight needles total per patient, was simulated for the IC/IS plans. The tandem IC applicator was shared for all treatment plans. Optimization and display of results was accomplished with an in-house treatment planning system.

For IC/IS an additional 30-70 minutes for interstitial needle placement, reconstruction in the treatment planning process, and treatment planning time beyond the IC approaches, which were all other approaches considered, was included in the delivery time calculation. The additional IC/IS time was included by adding 30 minutes to the delivery time required for fresh sources (10 Ci ¹⁹²Ir) and 70 minutes to the delivery time required for aged sources (4.3 Ci ¹⁹²Ir), resulting in best-case and worst-case IC/IS delivery times.

Following treatment plan optimization, dwell times were scaled uniformly to maximize HR-CTV D₉₀ (minimum dose to the hottest 90% of the HR-CTV) under the GEC-ESTRO (Groupe Europeen de Curietherapie in the European Society for Radiotherapy & Oncology) constraints that the bladder, rectum, and sigmoid colon D_{2cc}-values (minimum dose to the hottest 2 cm³ of the structure) must be less than or equal to 90, 75, and 75 Gy_{EQD2}, respectively. Equivalent dose in 2 Gy fractions (EQD2) was calculated using α/β values of 10 Gy for the HR-CTV and 3 Gy for the bladder, rectum, and sigmoid colon, and external beam radiotherapy (EBRT) dose of 1.8 Gy in 25 fractions (44 Gy_{EQD2} to HR-CTV) was included. Hereafter, when referring to cervical cancer results, D₉₀ will refer to HR-CTV D₉₀ unless otherwise specified, and dose will always be in EQD2 units unless otherwise specified.

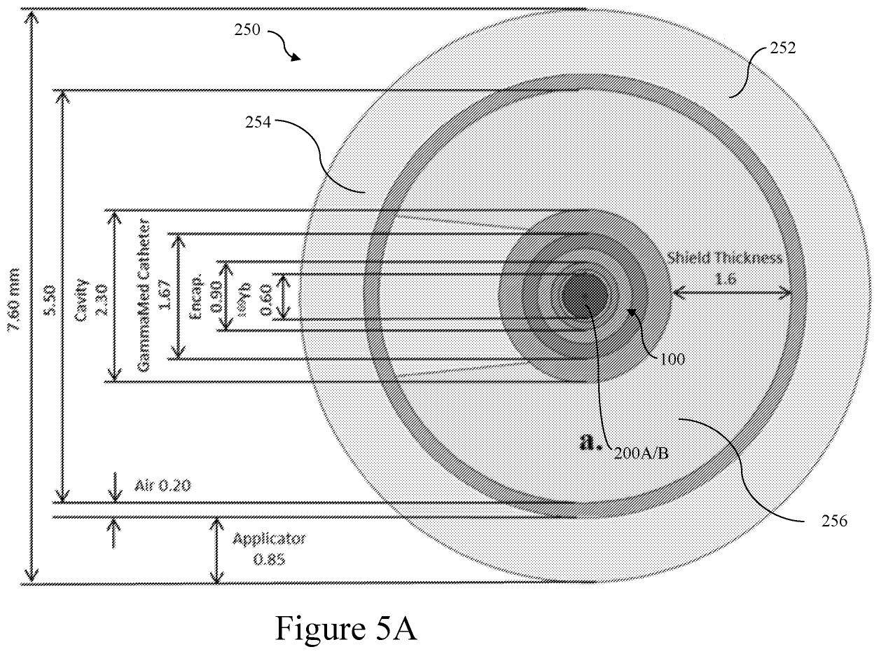

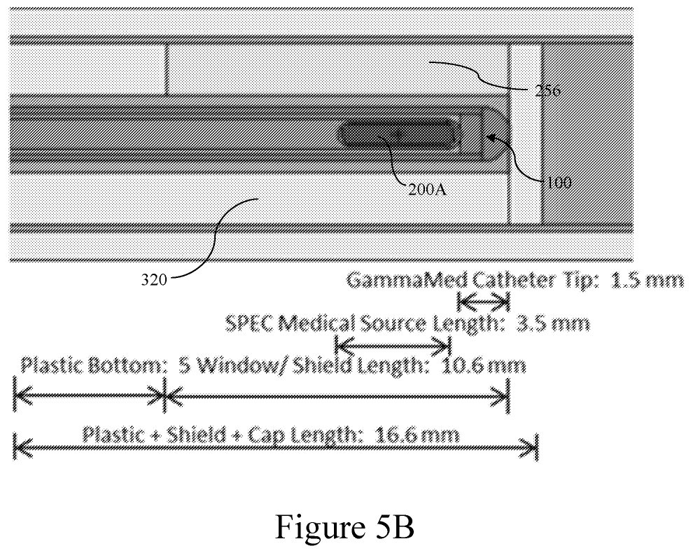

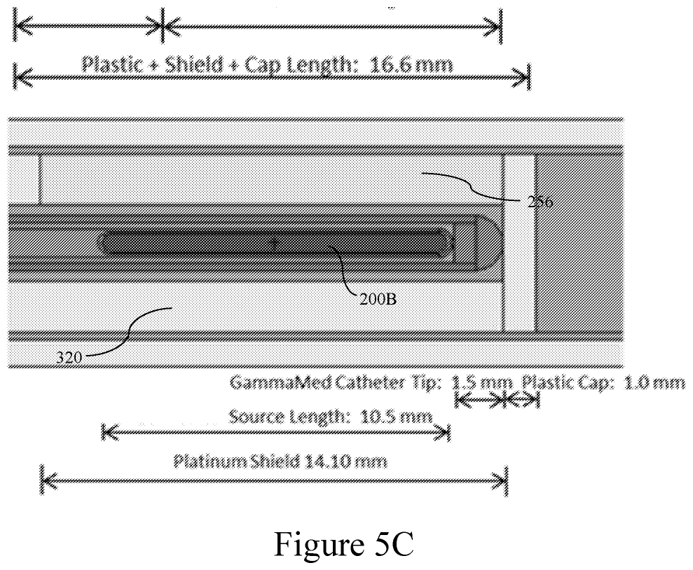

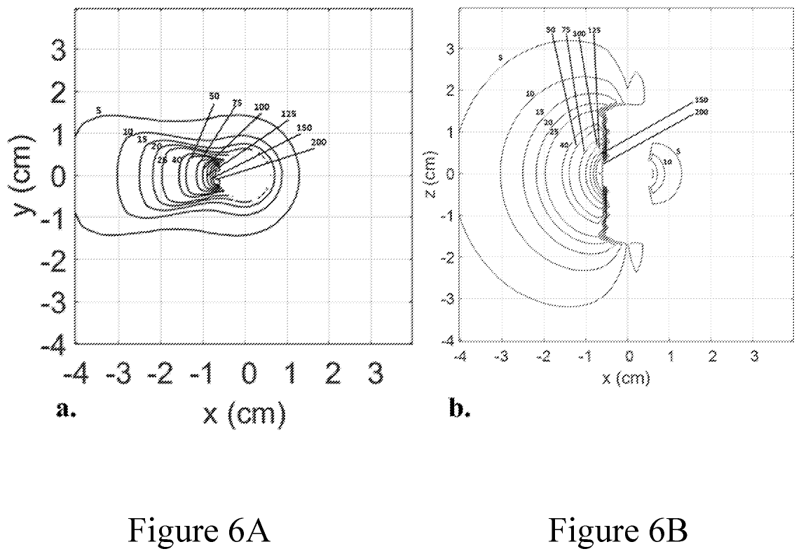

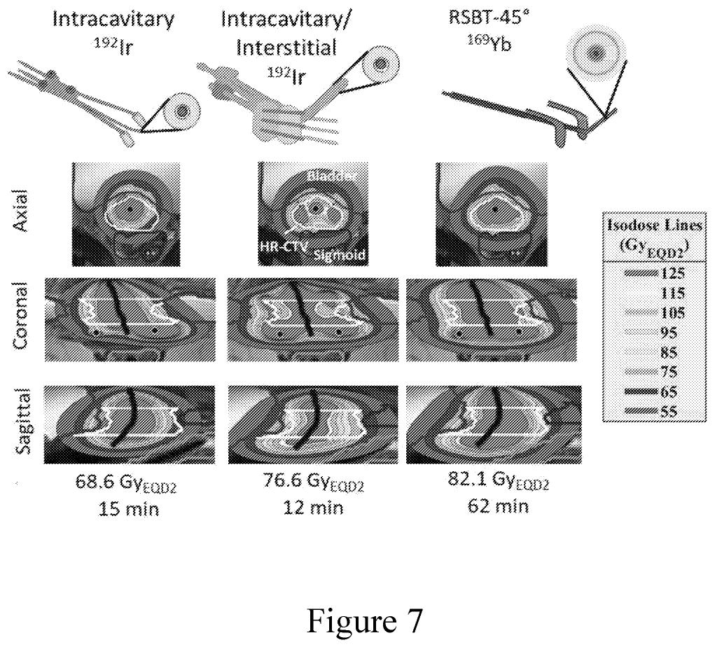

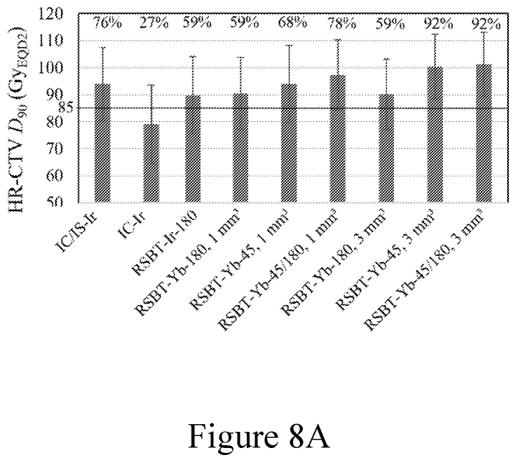

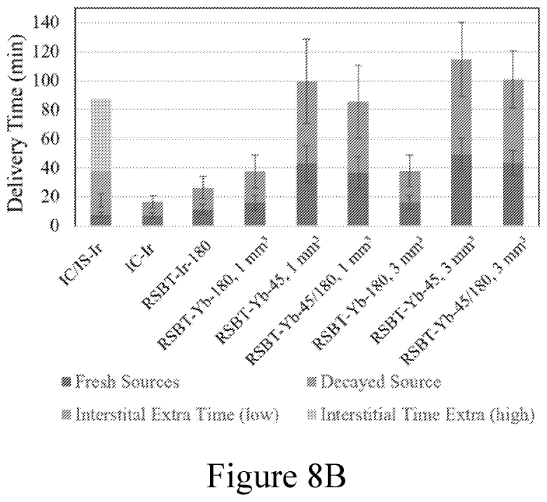

The cervical cancer tandem applicator model from MCNP used for RSBT-Yb-45 is shown for both the 1 mm³ and 3 mm³ active sources in FIGS. 5A-5C, and the normalized dose rate distributions generated from the model are shown in FIGS. 6A and 6B. More specifically, FIGS. 5A-5C illustrate Diagrams of cervical cancer RSBT-Yb-45 tandem applicator with versions for 1 mm³ and 3 mm^{3 169}Yb active sources, which have 0.6 mm active diameters and 3.5 mm and 10.5 mm active lengths, respectively. Green regions are platinum shields. FIG. 5A illustrates an Axial view for both 1 mm³ and 3 mm³ active sources; FIG. 5B illustrates a sagittal view for 1 mm³ active source 200A; and FIG. 5C illustrates a sagittal view for 3 mm³ active source 200B. The 1 mm^{3 169}Yb active source is based on SPEC M23 and Varian GammaMedplus sources. As shown in FIGS. 5A-5C, an applicator 250 can comprise a hollow body 252 defining a cavity 254 therethrough. A shield 256 can be inserted within the cavity 254, and the shield can have a concavity that receives a source 100. The shield 256 can block an angular sweep with respect to the hollow body's axis, thereby directing emitted radiation in a direction away from the shield 256, as should be understood. FIGS. 6A and 6B illustrate dose rate distributions for the 3 mm^{3 169}Yb radiation active source with an active source length diameter of 0.6 mm and an active source length of 10.5 mm for cervical cancer RSBT-Yb-45. Dose rate distributions were normalized to 100% at 1 cm from the source center in water on the unshielded side. FIG. 6A is an axial view from the applicator geometry in FIG. 5A; and FIG. 6B is a rotated sagittal view from the applicator geometry in FIG. 5C. Resulting dose distributions for IC, IC/IS, and RSBT-Yb-45° are shown in FIG. 7. Specifically, FIG. 7 illustrates dose distributions comparing ¹⁹²Ir-based IC HDR-BT (left column), ¹⁹²Ir-based IC/IS HDR-BT (middle column), and ¹⁶⁹Yb-based intracavitary RSBT delivered with a 45° emission angle. External beam radiation therapy dose of 1.8 Gy×25 fractions (44 Gy_{EQD2}) is included. Definitions: HDR-BT: high-dose-rate brachytherapy, IC=HDR-BT, IC/IS=intracavitary/interstitial HDR-BT, RSBT=rotating shield brachytherapy. Comprehensive mean and standard deviation D₉₀ and delivery times are shown in FIG. 8A and FIG. 8B, respectively, for all 37 cervical cancer patients. FIG. 8A illustrates HR-CTV D₉₀-values, including 44 Gy_{EQD2} of external beam radiotherapy dose, for RSBT techniques using either 1 mm³ or 3 mm^{3 169}Yb active sources, for 37 cervical cancer patients. Bars are the mean D₉₀-values and whiskers are one standard deviation. The 1 mm³ and 3 mm³ sources have active source diameters of 0.6 mm and lengths of 3.5 mm and 10.5 mm, respectively. The percentage of cases for which D₉₀ was 85 Gy_{EQD2} or greater is indicated for each delivery technique. FIG. 8B illustrates delivery times for the delivery techniques of FIG. 8A. For IC/IS, the low and high additional procedure time estimates required for interstitial needle insertion beyond IC (30-70 min) are included. Definitions: HR-CTV=high-risk clinical target volume; D₉₀=minimum dose to the hottest 90% of the HR-CTV; EQD2=equivalent dose delivered in 2 Gy fractions; IC/IS=combined intracavitary and interstitial high-dose-rate brachytherapy (HDR-BT) brachytherapy, IC=intracavitary HDR-BT; RSBT-(Ir/Yb)-(45/180)=rotating shield brachytherapy delivered using the ¹⁹²Ir (or ¹⁶⁹Yb) isotope with emission angles of 45°, 180°, or both. In addition to the D₉₀'s shown in FIG. 8A, the percentage of patients considered with D₉₀'s of ≥85 Gy, which is the goal HR-CTV dose for the EMBRACE II study, is listed above each bar. In FIG. 8A and FIG. 8B, it is shown that the current conventional HDR-BT options of IC and IC/IS, both based on the ¹⁹²Ir isotope, produced mean (±1 standard deviation) D₉₀'s of 79.1±14.4 Gy and 93.9±13.5 Gy, respectively, achieved D₉₀'s of ≥85 Gy in 27% and 76% of patients, respectively, with mean delivery times ranging from 7.2-16.7 min and 37.6-87.6 min, respectively. The mean D₉₀ dosimetric difference of 14.8 Gy_{EQD2} and the increase in percentage of patients for which D₉₀≥85 Gy_{EQD2} of 49 percentage points represents the clinical difference shown in RetroEMBRACE results, wherein local disease control at 3 years for HR-CTVs ≥30 cm³ was 92% for patients treated at centers using IC/IS (n=169) vs. 82% for patients treated at centers using IC only (n=118), with statistically equivalent grade 2-5 gastrointestinal and urinary bladder complication rates.

As shown in FIG. 8A, the ¹⁹²Ir-based RSBT-Ir-180 resulted in an HR-CTV D₉₀ of 89.8±14.4 Gy, with D₉₀'s of ≥85 Gy in 59% of patients. A slight improvement was obtained by switching from ¹⁹²Ir to ¹⁶⁹Yb. When using a 1 mm^{3 169}Yb active radiation source, RSBT-Yb-180 and RSBT-Yb-45 produced D₉₀'s of 90.5±13.3 Gy and 93.9±14.3 Gy, respectively, which were above 85 Gy for 59% and 68% of patients, respectively, and had fresh source delivery times of 16.0±4.8 min and 43±12.4 min, respectively. Combining the 180° and 45° emission angles resulted in further improvements that make RSBT-Yb-45/180 potentially superior to IC/IS, with D₉₀'s of 97.3±12.9 Gy, 78% of D₉₀'s≥85 Gy, and delivery times with fresh sources of 36.7±10.9 min. Over the full range of source ages, mean delivery times for RSBT-Yb-45/180 were 36.7-85.5 min, slightly shorter than the 37.6-87.6 min expected for IC/IS.

Increasing active source volume from 1 mm³ to 3 mm³ would have a major impact on ¹⁶⁹Yb source cost, with a 74-77% reduction in annual precursor and reactor time costs, and was found to actually improve the dosimetric results with an acceptable increase in delivery times. As shown in FIG. 8A, changing ¹⁶⁹Yb active source volume from 1 mm³ to 3 mm³ increased HR-CTV D₉₀-values for RSBT-Yb-45/180 to 101.2±11.9 Gy, a 3.9 Gy (4%) increase, and mean delivery times were 43.4-100.9 min. Since the HR-CTV D₉₀-values are so high for RSBT-Yb-45/180 and a common clinical goal is to achieve 85 Gy, dwell times could be scaled down to meet the 85 Gy goal, reducing delivery time substantially. The active source volume could be increased to 4 mm³ by holding the length at 10.5 and increasing the diameter to 0.69 mm, which would have a minimal additional impact on the delivered doses since active source length would be held constant, thus the 4 mm³ active source as described would be no more a line source than the 3 mm³ active source as described.

### 2. Prostate Cancer Dosimetric Considerations

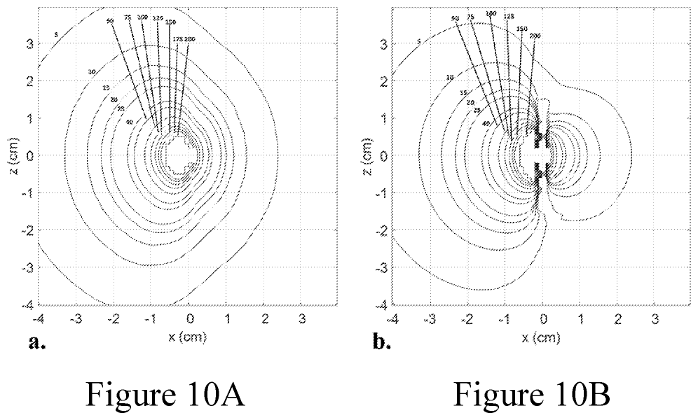

For prostate cancer, a dataset of 26 patients was considered, which was used in a previously-published study on the dosimetric effectiveness of RSBT for prostate cancer. All of the patients were clinically treated with HDR-BT, and radiation oncologists generated contours for the planning target volume (PTV), urethra, bladder, and rectum, which had mean (±one standard deviation) values of 61.7±14.3 cm³, 2.2±0.7 cm³, 60.6±28.7 cm³, and 42.1±14.9 cm³, respectively. The PTV was defined as the entire volume of the prostate with no additional margin added. A margin of 3 mm was added to the urethra contour to provide space for a dose gradient about the urethra. The clinically-used needle positions were used for the HDR-BT plans, and a median of 23 needles was used. FIGS. 9A-9C illustrate diagrams of prostate cancer RSBT nitinol needle 300 with versions for 1 mm³ and 3 mm^{3 169}Yb active sources, which have 0.6 mm active diameters and 3.5 mm and 10.5 mm active lengths, respectively. The needle can be hollow and define a generally cylindrical interior. An axially elongated platinum shield 302 can be received within the needle's interior via a catheter. As shown in FIG. 9A, the shield 302 can be disposed off-center with respect to the needle's central axis. The interior of the needle 300 can further define a cavity 306 to receive the source 100. As should be understood, the shield 302 can have a concavity with respect to its cross-section so that the shield 302 surrounds a portion of the source 100 along the axis of the source, thereby directing emitted radiation away from the shield 302. A platinum shield cap 304 can be disposed at a distal end of the needle. FIG. 9A is an axial view for both 1 mm³ and 3 mm³ active sources; FIG. 9B is a sagittal view for 1 mm³ active source; and FIG. 9C is a sagittal view for 3 mm³ active source. The 1 mm^{3 169}Yb active source is based on SPEC M23 and VARIAN GAMMAMEDPLUS sources. FIGS. 10A and 10B illustrates Dose rate distributions for the 3 mm^{3 169}Yb radiation active source with an active source length diameter of 0.6 mm and an active source length of 10.5 mm in a prostate cancer RSBT needle. Dose rate distributions were normalized to 100% at 1 cm from the source center in water on the unshielded side. FIG. 10A is an axial view from the applicator geometry in FIG. 9A; and FIG. 10B is a rotated sagittal view from the applicator geometry in FIG. 9C. Prostate RSBT needle models generated with MCNP are shown in FIGS. 9A-9C, and the resulting normalized dose rate distributions are shown in FIGS. 10A and 10B. For each patient, the needles were re-arranged for the RSBT plans, placing several needles within a few mm of the urethra. This enabled effective use of the sharp dose gradients produced by partial shielding of the ¹⁶⁹Yb for the RSBT plans, avoiding an unfair comparison with HDR-BT. Similarly, it was found important not to use the RSBT needle positions for the HDR-BT plans to avoid unfairly increasing urethra dose for those plans.

Two types of treatment plans for prostate RSBT were generated: dose escalation plans and urethra-sparing plans. The dose escalation plans represented one-shot RSBT monotherapy, in which patients are delivered as high a dose as possible in a single treatment fraction, with no other radiation therapy. Toxicity results for one-shot HDR-BT monotherapy have been quite low, with 0-2% rates of grade 3 or higher toxicity. Long-term (6+ year) biochemical relapse-free survival actuarial rates for one-shot HDR-BT for low-risk (n=22) and intermediate-risk (n=34) patients have recently been reported to be 82%±3%. These rates are inferior to the conventional treatments of multi-fraction HDR-BT, LDR-BT, and EBRT+brachytherapy, which have reported prostate-specific-antigen-free progression percentages of above 90% for 6 years post-treatment and beyond. It is well-known that increasing prostate dose increases biochemical relapse-free survival, but increasing dose can increases risk of complications. Prostate RSBT has the potential to dramatically increase radiation dose to the prostate without increasing dose to the bladder or rectum beyond their threshold doses and while holding urethra dose to the same levels as with HDR-BT monotherapy, which would be expected to improve cure rates without increasing toxicity rates. The rationale for considering dose escalation with prostate RSBT was thus to generate plans that would maximize biochemical relapse-free survival without increasing complication rates.

The urethra-sparing plans apply to the case of an HDR-BT boost delivered in addition to the EBRT dose, which is intended to increase the total dose delivered to the prostate beyond that possible with EBRT alone and may be more beneficial for patients with greater disease spread than would be expected in patients who would benefit from RSBT monotherapy. A limitation of combined EBRT and HDR-BT is that the genitourinary toxicity increases beyond that of EBRT alone, and a correlation has been reported between urethra D₁₀ (minimum dose to the hottest 10% of the urethra) and the rate of grade 2 or greater genitourinary toxicity, as well as decreased Expanded Prostate Cancer Index Composite urinary domain score. The rationale for RSBT in the context of urethra-sparing is to decrease urethra Dio while minimally compromising dose coverage of the remaining prostate, thus theoretically decreasing the toxicity of the combined EBRT and brachytherapy approach without decreasing the cure rate. Clinicians would not be restricted to this approach, as prostate dose escalation in the boost context could also be envisioned, or a combination of dose escalation and urethra-sparing relative to conventional HDR-BT.

In the treatment planning process, a hyaluronic acid spacer injection was simulated for all patients to model the displacement between the PTV and the rectum, such that the distance between the PTV and rectum was 1.5 cm. Conventional optimization parameters can be employed in the planning process, which can be based on the POGS approach. For the dose escalation plans, HDR-BT dose was prescribed such that 90% of the PTV received 110% of the prescribed physical dose of 20.5 Gy, and the final RSBT plan was created by scaling the dwell times to maximize the physical dose delivered without exceeding the urethra D₁₀ that was obtained from the HDR-BT plan for the same patient. For the urethra-sparing plans, HDR-BT dose was prescribed such that 90% of the PTV received 110% of the prescribed physical dose of 15 Gy, and the final RSBT plan was created by scaling the dwell times to minimize urethra D₁₀ while matching the PTV D₉₀ from the HDR-BT plan. For the dose escalation plans, the increase in PTV D₉₀ compared with HDR-BT was the metric for RSBT improvement because it reflects the magnitude of dose escalation to the PTV that is possible with RSBT. In the urethra-sparing plans, the decrease in urethra D₁₀ compared with HDR-BT was the metric for RSBT improvement because it reflects the reduction in dose to the urethra that is possible with RSBT. Urethra, bladder, and rectum dose tolerances were derived from previous clinical results for HDR-BT monotherapy, boost therapy, and cervical cancer brachytherapy. For dose escalation the urethra, bladder, and rectum tolerances were D₁₀<22.6 Gy, D_{2cc}<20.7 Gy, and D_{2cc}<18.5 Gy, respectively, and for urethra-sparing they were D₁₀<16.5 Gy, D_{2cc}<12.9 Gy, and D_{2cc}<17.1 Gy, respectively.

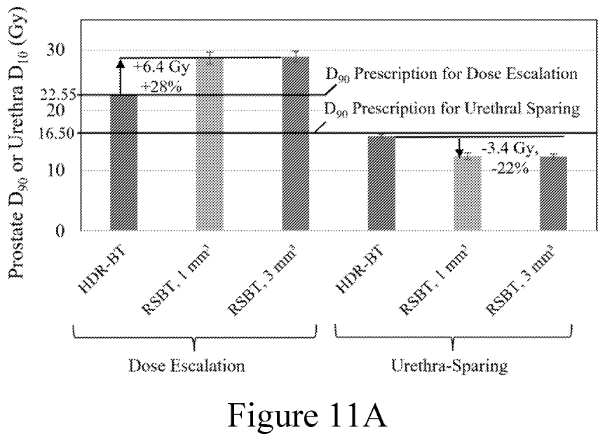

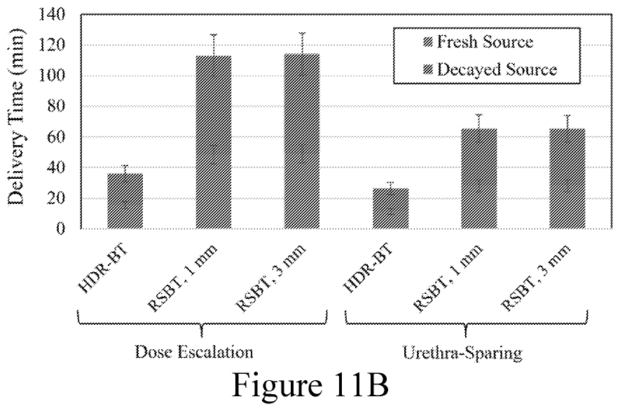

Comprehensive mean and standard deviation (over 26 patients) prostate D₉₀ and urethra D₁₀ values are shown in FIG. 11A, and delivery times are shown in FIG. 11B. FIG. 11A illustrates prostate mean (bars) and standard deviation (whiskers) D₉₀-values for single-shot dose escalation (monotherapy) and urethra D₁₀-values for single-shot urethra-sparing (boost therapy) for 26 patients. ¹⁶⁹Yb-based RSBT improved mean prostate D₉₀ by 6.4 Gy (28%) and reduced urethra D₁₀ by 2.4 Gy (22%) relative to conventional HDR-BT for dose escalation and urethra-sparing plans, respectively. The 1 mm³ and 3 mm³ active sources have active source diameters of 0.6 mm and lengths of 3.5 mm and 10.5 mm, respectively. FIG. 11B illustrates delivery times for the delivery techniques of FIG. 11A. As shown in both FIGS. 11A and 11B, dosimetric and treatment time results were equivalent for both 1 mm³ and 3 mm^{3 169}Yb active sources. Definitions for FIGS. 11A and 11B: D₉₀=minimum dose to the hottest 90% of the prostate; D₁₀=minimum dose to the hottest 10% of the urethra; HDR-BT=high-dose-rate brachytherapy delivered with ¹⁹²Ir, RSBT=rotating shield brachytherapy delivered with ¹⁶⁹Yb. As shown in FIG. 11A, the use of RSBT for prostate dose escalation in the monotherapy setting resulted in substantial dosimetric gains, with a mean (±1 standard deviation) prostate D₉₀ increase from 22.55±0.0 Gy for HDR-BT to 28.6±1.0 Gy for RSBT, a 6.4 Gy (28%) mean prostate D₉₀ increase with no increase in urethra D₁₀. Dose escalation delivery times for HDR-BT were 15.5±2.2 minutes (10.0 Ci of ¹⁹²Ir) to 36.0±5.2 minutes (4.3 Ci of ¹⁹²Ir, and delivery times for RSBT were longer at 48.6±5.9 minutes (27.0 Ci of ¹⁶⁹Yb) to 113.0±13.6 minutes (11.6 Ci of ¹⁶⁹Yb). RSBT provided a substantial dosimetric benefit for urethra-sparing, a urethra D₁₀ decrease from 15.7±0.3 Gy for HDR-BT to 12.4±0.5 Gy for RSBT, a 3.4 Gy (22%) reduction without compromising prostate D₉₀. Urethra-sparing RSBT delivery times for HDR-BT were 11.4±1.7 minutes (10.0 Ci of ¹⁹²Ir to 26.4±3.9 minutes (4.3 Ci of ¹⁹²Ir, and delivery times for RSBT were longer at 28.2±3.8 minutes (27.0 Ci of ¹⁶⁹Yb) to 65.4±8.8 minutes (11.6 Ci of ¹⁶⁹Yb). These dosimetric improvements also represent potentially clinically significant gains with feasible delivery times.

Increasing active source volume from 1 mm³ to 3 mm³ would have a major impact on ¹⁶⁹Yb source cost (74-77% reduction in annual precursor and reactor time costs), but a small impact on the dosimetric results and delivery times. As shown in FIGS. 11A and 11B, changing ¹⁶⁹Yb active source volume from 1 mm³ to 3 mm³ had a very small effect of 1% or less on mean prostate D₉₀, urethra D₁₀, and delivery times.

### M. Impact of Source Volume: Mechanical Considerations

Brachytherapy applicators in current clinical use for HDR-BT have in general been developed for use with active radiation sources with lengths of 3-5 mm, thus shorter than the 10.5 mm long active source shown in FIG. 1. A mechanical concern with active radiation sources of greater length is that their capability for use in conventional applicators may be mechanically limited due to their inability to navigate curves in existing applicators designed for use with shorter active sources. For example, a conventional tandem and ring applicator used for cervical cancer, such as the 3-D Interstitial Ring Applicator from Varian, has a ring diameter of 30 mm and a channel diameter that supports a 3.5 mm long active source, which is too small to support a 10.5 mm active radiation source without substantial modification of the channel diameter. According to one aspect, the ¹⁶⁹Yb active source disclosed herein is configured for use with applicators containing partial radiation shields. Accordingly, conventional applicators may be incompatible with the ¹⁶⁹Yb active source disclosed herein. Accommodating a ¹⁶⁹Yb active source that is 10.5 mm in length, for example, in a conventional tandem and ovoid applicator designed for cervical cancer would require an adjustment to the curvature of the ovoid applicator channels. Conventional tandem and ring applicators used for cervical cancer brachytherapy have an insufficiently small ring and channel to support the ¹⁶⁹Yb active source disclosed herein.

Applicators for other sites such as the breast, which can be treated effectively with a multi-curved-channel applicator such as SAVI (Cianna Medical, Aliso Viejo, Calif.) similarly are incompatible with ¹⁶⁹Yb active sources as disclosed herein. Such applicators can be replaced with an RSBT-type applicator having dimensions configured to receive the ¹⁶⁹Yb active source disclosed herein, which may deliver a dose distribution that is clinically equivalent to the SAVI applicator. The use of the ¹⁶⁹Yb active source disclosed herein in conjunction with a DMBT or RSBT applicator may be used to treat endometrial and vaginal cancer. According to some embodiments, a DMBT or RSBT applicator may be configured to have sufficient lack of curvature to receive the embodiments of the ¹⁶⁹Yb active sources as disclosed herein.

### Exemplary Aspects

In view of the described devices, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A ytterbium-169 source comprising: an active element having a volume between about two cubic millimeters and about four cubic millimeters, wherein the active element comprises between zero and thirty curies of ytterbium-169 at a start of an activation, wherein at an end of the activation, the active element will have a total activity of less than thirty curies and an activity concentration of less than ten curies per cubic millimeter.

Aspect 2: The ytterbium-169 source of aspect 1, wherein the active element has a length of between about 7.5 millimeters and about 10.5 millimeters.

Aspect 3: The ytterbium-169 source of aspect 2, wherein the active element has a length of between about 9 millimeters and about 10.5 millimeters.

Aspect 4: The ytterbium-169 source of aspect 2, wherein the active element has a length of between about 7.5 millimeters and about 9 millimeters.

Aspect 5: The ytterbium-169 source of aspect 1, wherein the active element has a diameter between about 0.60 and 0.69 millimeters.

Aspect 6: The ytterbium-169 source of aspect 5, wherein the active element has a diameter between about 0.60 and 0.65 millimeters.

Aspect 7: The ytterbium-169 source of aspect 5, wherein the active element has a diameter between about 0.65 and 0.69 millimeters.

Aspect 8: The ytterbium-169 source of aspect 1, wherein the active element has a diameter that is greater than 0.7 millimeters.

Aspect 9: The ytterbium-169 source of aspect 1, wherein the active element has a volume between 2.5 and 4 cubic millimeters.

Aspect 10: The ytterbium-169 source of aspect 9, wherein the active element has a volume between 2.8 and 4 cubic millimeters.

Aspect 11: The ytterbium-169 source of aspect 10, wherein the active element has a volume between 2.8 and 3.5 cubic millimeters.

Aspect 12: The ytterbium-169 source of aspect 9, wherein the active element has a volume between 2.5 and 3.5 cubic millimeters.

Aspect 13: The ytterbium-169 source of aspect 12, wherein the active element has a volume between 2.8 and 3.2 cubic millimeters.

Aspect 14: The ytterbium-169 source of aspect 10, wherein the active element has a volume between 3 and 4 cubic millimeters.

Aspect 15: The ytterbium-169 source of aspect 9, wherein the active element has a volume between 3.5 and 4 cubic millimeters.

Aspect 16: The ytterbium-169 source of any of the preceding aspects, further comprising a radiation source capsule and a radiation source wire, wherein the active element is disposed within the radiation source capsule, and wherein the radiation source wire is coupled to the radiation source capsule.

Aspect 17: The ytterbium-169 source of aspect 16, wherein the radiation source capsule is an outer capsule, wherein the ytterbium-169 source further comprises an inner capsule that surrounds the active element, wherein the inner capsule is disposed within the outer capsule.

Aspect 18: The ytterbium-169 source of aspect 16 or aspect 17, further comprising a disposable segment that couples the radiation source capsule to the radiation source wire, wherein the disposable segment is configured to be cut to decouple the radiation source wire from the radiation source capsule.

Aspect 19: The ytterbium-169 source of any one of aspects 16-18, wherein the radiation source wire is configured to be controlled by a remote afterloader.

Aspect 20: An applicator comprising any of the ytterbium-169 sources of aspects 1-19.

Aspect 21: The applicator as in aspect 20, wherein the applicator is a needle.

Aspect 22: A method comprising: using the ytterbium-169 source of any of aspects 1-19 in a brachytherapy treatment.

Aspect 23: A method for replacing a first active source with a second active source in a source assembly, the source assembly comprising a capsule having a hollow interior and an opening, a first active source disposed therein, and a retainer covering the opening and welded to the capsule, the method comprising: removing the weld connecting the retainer to the capsule; removing the retainer from the capsule; removing the first active source from the capsule; inserting a second active source in the capsule; replacing the retainer in the capsule; and welding the retainer to the capsule.

Aspect 24: The method of aspect 23, wherein the retainer comprises a plug and a receiver that is configured to couple to the capsule via weldment.

Aspect 25: A method comprising: irradiating an active element having an initial total activity between zero and thirty curies of ytterbium-169 and a volume between about two cubic millimeters and about four cubic millimeters; and ceasing irradiation before the active element surpasses a total activity of thirty curies and an activity concentration of ten curies per cubic millimeter.

Aspect 26: The method of aspect 25, wherein the active element has a length of between about 7.5 millimeters and about 10.5 millimeters.

Aspect 27: The method of aspect 26, wherein the active element has a length of between about 9 millimeters and about 10.5 millimeters.

Aspect 28: The method of aspect 26, wherein the active element has a length of between about 7.5 millimeters and about 9 millimeters.

Aspect 29: The method of aspect 25, wherein the active element has a diameter between about 0.60 and 0.69 millimeters.

Aspect 30: The method of aspect 29, wherein the active element has a diameter between about 0.60 and 0.65 millimeters.

Aspect 31: The method of aspect 29, wherein the active element has a diameter between about 0.65 and 0.69 millimeters.

Aspect 32: The method of aspect 1, wherein the active element has a diameter that is greater than 0.7 millimeters.

Aspect 33: The method of aspect 1, wherein the active element has a volume between 2.5 and 4 cubic millimeters.

Aspect 34: The method of aspect 33, wherein the active element has a volume between 2.8 and 4 cubic millimeters.

Aspect 35: The method of aspect 34, wherein the active element has a volume between 2.8 and 3.5 cubic millimeters.

Aspect 36: The method of aspect 33, wherein the active element has a volume between 2.5 and 3.5 cubic millimeters.

Aspect 37: The method of aspect 36, wherein the active element has a volume between 2.8 and 3.2 cubic millimeters.

Aspect 38: The method of aspect 34, wherein the active element has a volume between 3 and 4 cubic millimeters.

Aspect 39: The method of aspect 38, wherein the active element has a volume between 3.5 and 4 cubic millimeters.

Aspect 40: The method of aspect 25, further comprising: positioning the active element within a radiation source capsule; and coupling a radiation source wire to the radiation source capsule.

Aspect 41: The method of aspect 40, wherein irradiating the active element comprises irradiating the active element while the active element is in an inner capsule, wherein positioning the active element within the radiation source capsule comprises positioning the inner capsule within the radiation source capsule.

Aspect 42: The method of aspect 40, wherein coupling the radiation source wire to the radiation source capsule comprises coupling the radiation source wire to the radiation source capsule with a disposable segment that is configured to be cut to decouple the radiation source wire from the radiation source capsule.

Aspect 43: The method of aspect 42, further comprising: cutting the disposable segment to decouple the radiation source wire from the radiation source capsule.

Aspect 44: The method of aspect 40, further comprising establishing communication between the radiation source wire and a remote afterloader.

Aspect 45: The method of aspect 25, further comprising reactivating the active element after the step of ceasing irradiation, wherein reactivating the source comprises: irradiating the active source; and ceasing irradiation before the active element surpasses a total activity of thirty curies and an activity concentration of ten curies per cubic millimeter.

Aspect 46: A system comprising: an applicator; a catheter rotatably disposed within the applicator; and a ytterbium-169 source disposed within the catheter, wherein the ytterbium-169 source comprises: an active element having a volume between about two cubic millimeters and about four cubic millimeters, wherein the active element has a total activity of less than thirty curies and an activity concentration of less than ten curies per cubic millimeter.

Aspect 47: A method comprising: inserting, into an applicator, a ytterbium-169 source, wherein the ytterbium-169 source comprises: an active element having a volume between about two cubic millimeters and about four cubic millimeters, wherein the active element has a total activity of less than thirty curies and an activity concentration of less than ten curies per cubic millimeter.

Aspect 48: The method of aspect 47, further comprising: prior to inserting the ytterbium-169 source into the applicator, inserting a catheter into the applicator.

Aspect 49: The method of aspect 48, wherein inserting, into the applicator, the ytterbium-169 source comprises using an afterloader to insert the ytterbium-169 source into the catheter.

Aspect 50: The method of aspect 49, further comprising: rotating the catheter with respect to the applicator.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. A method comprising:
irradiating an active element having an initial total activity between zero and thirty curies of ytterbium-169 and a volume between about two cubic millimeters and about four cubic millimeters; and
ceasing irradiation before the active element surpasses a total activity of thirty curies and an activity concentration of ten curies per cubic millimeter.

2. The method of claim 1, wherein the active element has a length of between about 7.5 millimeters and about 10.5 millimeters.

3. (canceled)

4. (canceled)

5. The method of claim 1, wherein the active element has a diameter between about 0.60 and 0.69 millimeters.

6. (canceled)

7. (canceled)

8. The method of claim 1, wherein the active element has a diameter that is greater than 0.7 millimeters.

9. The method of claim 1, wherein the active element has a volume between 2.5 and 4 cubic millimeters.

10. (canceled)

11. (canceled)

12. The method of claim 9, wherein the active element has a volume between 2.5 and 3.5 cubic millimeters.

13. The method of claim 12, wherein the active element has a volume between 2.8 and 3.2 cubic millimeters.

14. The method of claim 9, wherein the active element has a volume between 3 and 4 cubic millimeters.

15. The method of claim 9, wherein the active element has a volume between 3.5 and 4 cubic millimeters.

16. The method of claim 1, further comprising:
positioning the active element within a radiation source capsule; and
coupling a radiation source wire to the radiation source capsule.

17. The method of claim 16, wherein irradiating the active element comprises irradiating the active element while the active element is in an inner capsule, wherein positioning the active element within the radiation source capsule comprises positioning the inner capsule within the radiation source capsule.

18. The method of claim 16, wherein coupling the radiation source wire to the radiation source capsule comprises coupling the radiation source wire to the radiation source capsule with a disposable segment that is configured to be cut to decouple the radiation source wire from the radiation source capsule.

19. The method of claim 18, further comprising: cutting the disposable segment to decouple the radiation source wire from the radiation source capsule.

20. The method of claim 16, further comprising establishing communication between the radiation source wire and a remote afterloader.

21. The method of claim 1, further comprising reactivating the active element after the step of ceasing irradiation, wherein reactivating the source comprises:
irradiating the active source; and
ceasing irradiation before the active element surpasses a total activity of thirty curies and an activity concentration of ten curies per cubic millimeter.

22. A system comprising:
an applicator;
a catheter rotatably disposed within the applicator; and
a ytterbium-169 source disposed within the catheter, wherein the ytterbium-169 source comprises:
an active element having a volume between about two cubic millimeters and about four cubic millimeters,
wherein the active element has a total activity of less than thirty curies and an activity concentration of less than ten curies per cubic millimeter.

23. A method comprising:
inserting, into an applicator, a ytterbium-169 source, wherein the ytterbium-169 source comprises:
an active element having a volume between about two cubic millimeters and about four cubic millimeters,
wherein the active element has a total activity of less than thirty curies and an activity concentration of less than ten curies per cubic millimeter.

24. The method of claim 23, further comprising: prior to inserting the ytterbium-169 source into the applicator, inserting a catheter into the applicator.

25. The method of claim 24, wherein inserting, into the applicator, the ytterbium-169 source comprises using an afterloader to insert the ytterbium-169 source into the catheter.

26. The method of claim 25, further comprising: rotating the catheter with respect to the applicator.
